(19) United States
(12) Patent Application Publication
(10) Pub. No.: US 2024/0033329 A1
(43) Pub. Date: Feb. 1, 2024

(54) COMPOSITIONS AND METHODS FOR CONTROLLED OVARIAN STIMULATION

(71) Applicant: Ferring B.V., Hoofddorp (NL)

(72) Inventor: Joan-Carles ARCE, Hoofddorp (NL)

(73) Assignee: Ferring B.V., Hoofddorp (NL)

(21) Appl. No.: 18/266,159

(22) PCT Filed: Dec. 9, 2021
(86) PCT No.: PCT/EP2021/084958

§ 371 Date: Jun. 8, 2023

(30) Foreign Application Priority Data
Dec. 9, 2020 (EP) 20212885.6
Jan. 11, 2021 (EP) 21150909.6
Feb. 12, 2021 (EP) 21156870.4

(51) Int. Cl. A61K 38/24; A61P 15/08

(52) U.S. Cl. CPC A61K 38/24; A61P 15/08

(57) ABSTRACT

Methods using and compositions including FSH for use in the treatment of infertility are described.

The present invention relates to methods, compositions and pharmaceutical products for the treatment of infertility.

### BACKGROUND

Assisted reproductive technologies (ART) such as in vitro fertilisation (IVF) and microinsemination are well known. ART generally requires a step of controlled ovarian stimulation (COS), in which a cohort of follicles is stimulated to full maturity. Standard COS regimens include administration of gonadotrophins, such as follicle stimulating hormone (FSH), alone or in combination with luteinising hormone (LH) activity to stimulate multiple follicular development. Usually COS requires administration of a GnRH analogue (GnRH agonist) or GnRH antagonist prior to and/or during stimulation to prevent a premature LH surge which may induce ovulation before planned oocyte retrieval. The pharmaceutical compositions generally used for COS include recombinant follicle stimulating hormone (rFSH) including REKOVELLE® and GONAL-F®, urinary derived FSH, recombinant FSH+LH preparations, urinary derived menotrophin [human menopausal gonadotrophin (hMG)] and highly purified human menopausal gonadotrophin (HP-hMG).

In case of a too high ovarian response, COS can be associated with a risk of ovarian hyperstimulation syndrome (OHSS), which can lead to cancellation of the COS cycle and can become life threatening in severe cases. The ability to predict the ovarian response potential of women to COS may allow the development of personalised or individualised COS protocols. Such individualised protocols could, for example, reduce the risk of OHSS in women predicted to have an excessive ovarian response to COS, and prevent cancellation of COS cycles. Levels of Anti Mullerian Hormone (AMH) are directly correlated with the ovarian response to gonadotrophins during COS. Thus, high levels of AMH are a good predictor of excessive ovarian response, and an indicator of risk of OHSS, whereas low levels of AMH predict a poor ovarian response to COS.

Clinical research has focused the last years on the development of individualised dosing regimens for COS, initially without using AMH but based on other predictors of ovarian response. These predictors include age, body mass index (BMI), FSH, and antral follicle count (AFC).

As indicated above, standard COS protocols require daily FSH administration to induce multiple follicular growth to obtain sufficient oocytes for IVF. FSH is a natural hormone that is secreted by the anterior pituitary gland. In healthy women FSH induces monthly the growth of a single dominant follicle that ovulates during each natural cycle. FSH purified from the urine of post-menopausal women has been used for many years in infertility treatment, both to promote ovulation in natural reproduction and to induce multiple follicular growth to obtain sufficient oocytes for ART.

Until recently, the only approved rFSH products for ovarian stimulation, such as follitropin alfa (GONAL-F®, Merck Serono/EMD Serono) and follitropin beta (PUREGON®/FOLLISTIM®, MSD/Schering-Plough), were derived from a Chinese Hamster Ovary (CHO) cell line. The present applicants have developed a human cell line-derived rFSH which is the subject of International Patent Application No. PCT/GB2009/000978, published as WO2009/127826A. On 13 Dec. 2016, the European Commission (EC) granted marketing authorisation for REKOVELLE® (follitropin delta, also known as FE 999049), a human cell line-derived recombinant follicle stimulating hormone (human rFSH), for use in controlled ovarian stimulation for the development of multiple follicles in women undergoing assisted reproductive technologies (ART), such as an in vitro fertilisation (IVF) cycle. REKOVELLE® is the first rFSH to be derived from a human cell line. The REKOVELLE© (follitropin delta) product is produced by the methods disclosed in International Patent Application No. PCT/GB2009/000978.

The posology of REKOVELLE® is individualised for each patient and aims to obtain an ovarian response which is associated with a favourable safety/efficacy profile, i.e. aims to achieve an adequate number of oocytes retrieved and reduce the interventions to prevent OHSS. REKOVELLE® is dosed in micrograms (μg). For the first treatment cycle, the individual daily dose is determined on the basis of the woman's serum AMH concentration and, depending on serum AMH concentration, her body weight. The dose is based on a recent determination of AMH (i.e. within the last 12 months) measured by the ELECSYS® AMH Plus immunoassay (Roche). The individual daily dose is maintained throughout the stimulation period. For women with AMH<15 pmol/L the daily dose of REKOVELLE® is 12 μg, irrespective of body weight. For women with AMH≥15 pmol/L the daily dose of REKOVELLE® is lower, and ranges from 0.19 μg/kg to 0.10 μg/kg over AMH concentrations of 15 to ≥40 pmol/L. The maximum dose in the first cycle is 12 μg. For subsequent treatment cycles, the daily dose of REKOVELLE® is maintained or may be modified according to the patient's ovarian response in the previous cycle. If the patient had adequate ovarian response in the previous cycle without developing OHSS, the same daily dose is used. In case of ovarian hypo-response in the previous cycle, the daily dose in the subsequent cycle is increased by 25% or 50%, according to the extent of response observed. In case of ovarian hyper-response in the previous cycle, the daily dose in the subsequent cycle is decreased by 20% or 33%, according to the extent of response observed. In patients who developed OHSS or were at risk of OHSS in a previous cycle, the daily dose for the subsequent cycle is 33% lower than the dose the cycle where OHSS or risk of OHSS occurred. The maximum daily dose of REKOVELLE® is 24 μg.

Still, there is a need for individualised COS protocols which provide improved pregnancy outcomes.

### SUMMARY

In a trial comparing REKOVELLE® with a CHO cell derived rFSH, the applicants unexpectedly found that early pregnancy loss was reported for 20% of subjects with a positive βhCG test treated with REKOVELLE®, compared to 34.6% of subjects with a positive βhCG test treated with the CHO cell derived rFSH (Example 1). Similarly, the Examples have shown that REKOVELLE® treatment increases the probability of a live birth, compared to treatment with a CHO cell derived rFSH. In one example, the improvement in live birth rate seen with REKOVELLE® is greater in the higher age strata (e.g. patients of age 28 or over, for example age 30 or over, for example age 38 or over).

In accordance with some aspects, there are provided compositions comprising recombinant follicle stimulating hormone (FSH) for use in reducing the likelihood of early pregnancy loss in a patient treated for infertility by controlled ovarian stimulation, wherein the recombinant FSH includes α2,3- and α2,6-sialylation (e.g. wherein 1% to 60% of the total sialylation is α2,6-sialylation and wherein 40% to 99% of the total sialylation is α2,3-sialylation, e.g. wherein 5% to 20% of the total sialylation is α2,6-sialylation and wherein 80% to 95% of the total sialylation is α2,3-sialylation). In accordance with some aspects, there are provided compositions comprising recombinant follicle stimulating hormone (FSH) for use in increasing the probability of live birth following treatment for infertility by controlled ovarian stimulation, wherein the recombinant FSH includes α2,3- and α2,6-sialylation (e.g. wherein 1% to 60% of the total sialylation is α2,6-sialylation and wherein 40% to 99% of the total sialylation is α2,3-sialylation, e.g. wherein 5% to 20% of the total sialylation is α2,6-sialylation and wherein 80% to 95% of the total sialylation is α2,3-sialylation). In accordance with some aspects, there are provided compositions comprising recombinant follicle stimulating hormone (FSH) for use in the treatment of infertility (e.g. by controlled ovarian stimulation) in a female patient of age 35 years or greater, for example of age 36 years or greater, for example of age 37 years or greater, for example of age 38 years or greater, for example a female patient of age 38-40 years, to increase the probability of live birth, wherein the recombinant FSH includes α2,3- and α2,6-sialylation (e.g. wherein 1% to 60% of the total sialylation is α2,6-sialylation and wherein 40% to 99% of the total sialylation is α2,3-sialylation, e.g. wherein 5% to 20% of the total sialylation is α2,6-sialylation and wherein 80% to 95% of the total sialylation is α2,3-sialylation). In some aspects, the use may comprise a step of determining the age of the patient, and a step of administering the recombinant FSH to a patient of age 35 years or greater, for example of age 36 years or greater, for example of age 37 years or greater, for example of age 38 years or greater, for example a female patient of age 38-40 years (to increase thereby the probability of live birth). In accordance with some aspects, there are provided compositions comprising recombinant follicle stimulating hormone (FSH) for use in the treatment of infertility (e.g. by controlled ovarian stimulation) in a female patient of age 28 years or greater, for example of age 30 years or greater, for example of age 31 years or greater, for example of age 32 years or greater, for example of age 33 years or greater, for example of age 35 years or greater, for example of age 36 years or greater, for example of age 37 years or greater, for example of age 38 years or greater, for example a female patient of age 28-40 years, for example a female patient of age 30-40 years, to increase the probability of live birth, wherein the recombinant FSH includes α2,3- and α2,6-sialylation (e.g. wherein 1% to 60% of the total sialylation is α2,6-sialylation and wherein 40% to 99% of the total sialylation is α2,3-sialylation, e.g. wherein 5% to 20% of the total sialylation is α2,6-sialylation and wherein 80% to 95% of the total sialylation is α2,3-sialylation). In some aspects, the use may comprise a step of determining the age of the patient, and a step of administering the recombinant FSH to a patient of age 28 years or greater, for example of age 30 years or greater, for example a female patient of age 28-40 years, for example a female patient of age 30-40 years. In aspects, the patient may have serum AMH level of <15 pmol/L, wherein the composition is to be administered at a dose of, or equivalent to, 11 to 13 μg recombinant FSH per day. The use may comprise a step of determining the serum AMH level of the patient, and a step of administering the dose to a patient having serum AMH level of <15 pmol/L. In some aspects, the patient may have serum AMH level of ≥15 pmol/L, wherein the composition is to be administered at a dose of, or equivalent to, 0.09 to 0.19 μg recombinant FSH per kg bodyweight of the patient per day. The use may comprise a step of determining the serum AMH level of the patient, and a step of administering the dose to a patient having serum AMH level of ≥15 pmol/L.

In accordance with some aspects, there are provided compositions comprising recombinant follicle stimulating hormone (FSH) for use in the treatment of infertility in a female patient of age 30 to 40 years, to increase the probability of live birth, wherein the recombinant FSH includes α2,3- and α2,6-sialylation. In some aspects, the use may comprise a step of determining the age of the patient, and a step of administering the recombinant FSH to a patient of age 30-40 years. In accordance with some aspects, there are provided compositions comprising recombinant follicle stimulating hormone (FSH) for use in the treatment of infertility in a female patient of age 30 to 37 years, to increase the probability of live birth, wherein the recombinant FSH includes α2,3- and α2,6-sialylation. In some aspects, the use may comprise a step of determining the age of the patient, and a step of administering the recombinant FSH to a patient of age 30-37 years. In accordance with some aspects, there are provided compositions comprising recombinant follicle stimulating hormone (FSH) for use in the treatment of infertility in a female patient of age 36 to 40 years, to increase the probability of live birth, wherein the recombinant FSH includes α2,3- and α2,6-sialylation. In some aspects, the use may comprise a step of determining the age of the patient, and a step of administering the recombinant FSH to a patient of age 36-40 years. In aspects, the patient may have serum AMH level of <15 pmol/L, wherein the composition is to be administered at a dose of, or equivalent to, 11 to 13 μg recombinant FSH per day. The use may comprise a step of determining the serum AMH level of the patient, and a step of administering the dose to a patient having serum AMH level of <15 pmol/L. In some aspects, the patient may have serum AMH level of ≥15 pmol/L, wherein the composition is to be administered at a dose of, or equivalent to, 0.09 to 0.19 μg recombinant FSH per kg bodyweight of the patient per day. The use may comprise a step of determining the serum AMH level of the patient, and a step of administering the dose to a patient having serum AMH level of ≥15 pmol/L. In accordance with some aspects, there are provided compositions comprising recombinant follicle stimulating hormone (FSH) for use in the treatment of infertility in a female patient of age 35 to 40 years, to increase the probability of live birth, wherein the recombinant FSH includes α2,3- and α2,6-sialylation, wherein if the patient has serum AMH level of <15 pmol/L, the composition is to be administered at a dose of, or equivalent to, 11 to 13 μg recombinant FSH per day, and if the patient has serum AMH level of ≥15 pmol/L, the composition is to be administered at a dose of, or equivalent to, 0.09 to 0.19 μg recombinant FSH per kg bodyweight of the patient per day. In some aspects, the use may comprise a step of determining the age of the patient, and a step of administering the recombinant FSH to a patient of age 35-40 years. In some aspects, the use may comprise a step of determining the serum AMH level of the patient, and a step of administering the specified dose to a patient having the specified serum AMH level. In some aspects the patient may be (for example may be identified as being) a female of Japanese ethnicity, for example a female with two ethnic Japanese parents. In some aspects the patient may be (for example may be identified as being) a female of Asian (e.g. Chinese, Taiwanese, Vietnamese or South Korean) ethnicity, for example a patient with two ethnic Asian (e.g. Chinese, Taiwanese, Vietnamese or South Korean) parents. In accordance with some aspects, there are provided compositions comprising recombinant follicle stimulating hormone (FSH) for use in the treatment of infertility in a female patient of age 35 years or greater, for example of age 36 years or greater, to increase the probability of live birth, wherein the recombinant FSH includes α2,3- and α2,6-sialylation, and wherein the patient is of Japanese or Asian ethnicity. In some aspects, the use comprises a step of determining the age of the patient, and a step of administering the recombinant FSH to a patient of age 35 years or greater, for example of age 36 years or greater. In aspects, the recombinant FSH is an FSH wherein 1% to 60% of the total sialylation of the FSH is α2,6-sialylation and 40% to 99% of the total sialylation of the FSH is α2,3-sialylation, e.g. wherein 5% to 20% of the total sialylation is α2,6-sialylation and wherein 80% to 95% of the total sialylation is α2,3-sialylation). In aspects, the recombinant FSH includes α2,3- and α2,6-sialylation wherein 5% to 20% of the total sialylation is α2,6-sialylation and wherein 80% to 95% of the total sialylation is α2,3-sialylation. In aspects, the recombinant FSH is recombinant FSH which has been produced or expressed in a human cell line.

In accordance with some aspects, there are provided methods of reducing the likelihood of early pregnancy loss in a patient treated for infertility by controlled ovarian stimulation, the methods comprising administering to the patient a pharmaceutically effective amount of a composition comprising recombinant FSH; wherein the recombinant FSH includes α2,3- and α2,6-sialylation (e.g. wherein 1% to 60% of the total sialylation is α2,6-sialylation and wherein 40% to 99% of the total sialylation is α2,3-sialylation, e.g. wherein 5% to 20% of the total sialylation is α2,6-sialylation and wherein 80% to 95% of the total sialylation is α2,3-sialylation). In accordance with some aspects, there are provided methods of increasing the probability of live birth following treatment of a patient for infertility by controlled ovarian stimulation, the methods comprising administering to the patient a pharmaceutically effective amount of a composition comprising recombinant FSH; wherein the recombinant FSH includes α2,3- and α2,6-sialylation (e.g. wherein 5% to 20% of the total sialylation is α2,6-sialylation and wherein 80% to 95% of the total sialylation is α2,3-sialylation). In accordance with some aspects, there are provided methods of increasing the probability of live birth following treatment of infertility (e.g. by controlled ovarian stimulation) of a female patient of age 35 years or greater, for example of age 36 years or greater, for example of age 37 years or greater, for example of age 38 years or greater, for example a female patient of age 38-40 years, the methods comprising administering to the patient a pharmaceutically effective amount of a composition comprising recombinant FSH; wherein the recombinant FSH includes α2,3- and α2,6-sialylation (e.g. wherein 5% to 20% of the total sialylation is α2,6-sialylation and wherein 80% to 95% of the total sialylation is α2,3-sialylation). In some aspects the methods may comprise a step of determining the age of the patient, and a step of administering the pharmaceutically effective amount of the composition comprising recombinant FSH to a patient of age 35 years or greater, for example of age 36 years or greater, for example of age 37 years or greater, for example of age 38 years or greater, for example a female patient of age 38-40 years. In accordance with some aspects, there are provided methods of treatment of infertility (e.g. by controlled ovarian stimulation, e.g. to increase the probability of live birth) in a female patient of age 35 years or greater, for example of age 36 years or greater, for example of age 37 years or greater, for example of age 38 years or greater, for example a female patient of age 38-40 years, the methods comprising a step of determining the age of the patient, and a step of administering a pharmaceutically effective amount of a composition comprising recombinant FSH to a patient of age 35 years or greater, for example of age 36 years or greater, for example of age 37 years or greater, for example of age 38 years or greater, for example a female patient of age 38-40 years; wherein the recombinant FSH includes α2,3- and α2,6-sialylation (e.g. wherein 5% to 20% of the total sialylation is α2,6-sialylation and wherein 80% to 95% of the total sialylation is α2,3-sialylation). In accordance with some aspects, there are provided methods of increasing the probability of live birth following treatment of infertility (e.g. by controlled ovarian stimulation) of a female patient of age 28 years or greater, for example of age 30 years or greater, for example of age 31 years or greater, for example of age 32 years or greater, for example of age 33 years or greater, for example of age 35 years or greater, for example of age 36 years or greater, for example of age 37 years or greater, for example of age 38 years or greater, for example a female patient of age 28-40 years, for example a female patient of age 30-40 years, the methods comprising administering to the patient a pharmaceutically effective amount of a composition comprising recombinant FSH; wherein the recombinant FSH includes α2,3- and α2,6-sialylation (e.g. wherein 5% to 20% of the total sialylation is α2,6-sialylation and wherein 80% to 95% of the total sialylation is α2,3-sialylation). In some aspects the methods may comprise a step of determining the age of the patient, and a step of administering the pharmaceutically effective amount of the composition comprising recombinant FSH to the patient of age 28 years or greater, for example of age 30 years or greater, for example of age 31 years or greater, for example of age 32 years or greater, for example of age 33 years or greater, for example of age 35 years or greater, for example of age 36 years or greater, for example of age 37 years or greater, for example of age 38 years or greater, for example a female patient of age 28-40 years, for example a female patient of age 30-40 years. In accordance with some aspects, there are provided methods of treatment of infertility (e.g. by controlled ovarian stimulation, e.g. to increase the probability of live birth) in a female patient of age 28 years or greater, for example of age 30 years or greater, for example of age 31 years or greater, for example of age 32 years or greater, for example of age 33 years or greater, for example of age 35 years or greater, for example of age 36 years or greater, for example of age 37 years or greater, for example of age 38 years or greater, for example a female patient of age 28-40 years, for example a female patient of age 30-40 years, the methods comprising a step of determining the age of the patient, and a step of administering a pharmaceutically effective amount of a composition comprising recombinant FSH to the patient of age 28 years or greater, for example of age 30 years or greater, for example of age 31 years or greater, for example of age 32 years or greater, for example of age 33 years or greater, for example of age 35 years or greater, for example of age 36 years or greater, for example of age 37 years or greater, for example of age 38 years or greater, for example a female patient of age 28-40 years, for example a female patient of age 30-40 years; wherein the recombinant FSH includes α2,3- and α2,6-sialylation (e.g. wherein 5% to 20% of the total sialylation is α2,6-sialylation and wherein 80% to 95% of the total sialylation is α2,3-sialylation). In aspects, the patient may have serum AMH level of <15 pmol/L, wherein the composition is to be administered at a dose of, or equivalent to, 11 to 13 μg recombinant FSH per day. The method may comprise a step of determining the serum AMH level of the patient, and a step of administering the dose to a patient having serum AMH level of <15 pmol/L. In some aspects, the patient may have serum AMH level of ≥15 pmol/L, wherein the composition is to be administered at a dose of, or equivalent to, 0.09 to 0.19 μg recombinant FSH per kg bodyweight of the patient per day. The method may comprise a step of determining the serum AMH level of the patient, and a step of administering the dose to a patient having serum AMH level of ≥15 pmol/L. The patient may be (for example is identified as being) a female of Japanese ethnicity, for example a female with two ethnic Japanese parents.

### DETAILED DESCRIPTION

The present applicants have found that FSH including α2,3- and α2,6-sialylation (e.g. wherein 5% to 20% of the total sialylation is α2,6-sialylation and wherein 80% to 95% of the total sialylation is α2,3-sialylation) may be used to reduce early pregnancy loss and/or increase the probability of live birth in a patient treated for infertility, compared to treatment with a CHO cell derived product. These results are remarkable and unexpected.

Therefore, provided herein are treatments and dosing regimens constructed to reduce early pregnancy loss and/or increase the probability of live birth in a patient treated for infertility.

### Definitions

Technical and scientific terms used herein have the meanings commonly understood by one of ordinary skill in the art of assisted reproductive technology to which the present invention pertains, unless otherwise defined. Reference is made herein to various methodologies known to those of ordinary skill in the art. Any suitable materials and/or methods known to those of ordinary skill in the art can be utilized in carrying out the present invention. However, specific materials and methods are described. Materials, reagents and the like to which reference is made in the following description and examples are obtainable from commercial sources, unless otherwise noted.

It is to be understood, that any definitions and terms herein defined is meant to have the same meaning and purpose in any of the aspects and embodiments of the invention unless explicitly otherwise stated not to.

As used herein, the singular forms “a,” “an,” and “the” designate both the singular and the plural, unless expressly stated to designate the singular only.

As used herein, the term “about” means that the number or range is not limited to the exact number or range set forth, but encompass ranges around the recited number or range as will be understood by persons of ordinary skill in the art depending on the context in which the number or range is used. Unless otherwise apparent from the context or convention in the art, “about” mean up to plus or minus 10% of the particular term.

Herein the terms “patient” and “subject” and “female” and “woman” are used interchangeably.

A subject may have normal serum FSH level of 1 to 16 IU/L, for example 1 to 15 IU/L, for example 1 to 12 IU/L in the early follicular phase. Thus a composition or medicament as described herein may be for (use in) the treatment of infertility (and/or for controlled ovarian stimulation) in a subject having (or identified as having) normal serum FSH level of 1 to 16 IU/L, for example 1 to 15 IU/L, for example 1 to 12 IU/L in the early follicular phase. Serum FSH may be measured by methods well known in the art to identify the patient for treatment.

A subject may have a BMI>15 and BMI<40 kg/m², for example a BMI>17.5 and BMI<38 kg/m², for example a BMI>18 and BMI<25 kg/m², for example a BMI>20 and BMI<25 kg/m². Thus a composition or method as described herein may reduce early pregnancy loss and/or increase the probability of live birth in a patient treated for infertility having BMI>1 and BMI<40 kg/m², for example a subject having BMI>17.5 and BMI<38 kg/m², for example a subject having BMI>18 and BMI<25 kg/m², for example a subject having BMI>20 and BMI<25 kg/m². Thus a composition or method as described herein may reduce early pregnancy loss and/or increase the probability of live birth in a patient treated for infertility having BMI>17.5 and BMI<32 kg/m². BMI may be measured by methods well known in the art to identify the patient for treatment.

Herein the term “treatment of infertility” includes treatment of infertility by controlled ovarian stimulation (COS) or methods which include a step or stage of controlled ovarian stimulation (COS), for example in vitro fertilisation (IVF), or intracytoplasmic sperm injection (ICSI). The term “treatment of infertility” includes treatment of infertility in a subject having tubal or unexplained infertility, including treatment of infertility in a subject having endometriosis, for example stage I or stage II endometriosis, and/or in a subject with a partner with male factor infertility. The composition may be for (use in) the treatment of infertility (and/or for controlled ovarian stimulation) in a subject having endometriosis, for example in a subject having stage I or stage II endometriosis, as defined by The American Society for Reproductive Medicine (ASRM) classification system for the various stages of endometriosis, (stage IV most severe; stage I least severe) [American Society for Reproductive Medicine. Revised American Society for Reproductive Medicine classification of endometriosis: 1996. Fertil Steril 1997; 67,817 821.].

Herein the term “GnRH agonist” means gonadotropin-releasing hormone agonist. GnRH agonists are a class of medications that act as agonists of the gonadotropin-releasing hormone receptor (GnRH receptor), the biological target of gonadotropin-releasing hormone.

Herein the term “GnRH antagonist” means gonadotropin-releasing hormone antagonist. GnRH antagonists are a class of medications that antagonize the gonadotropin-releasing hormone receptor (GnRH receptor) and thus the action of gonadotropin-releasing hormone (GnRH).

The serum concentration of anti-Mullerian hormone (AMH) is now established as a reliable marker of ovarian reserve. Decreasing levels of AMH are correlated with reduced ovarian response to gonadotrophins during COS. Further, high levels of AMH are a good predictor of excessive ovarian response, and an indicator of risk of OHSS. For the first (and in some cases subsequent) treatment cycle, the individual daily dose may be determined on the basis of the woman's serum AMH concentration and, depending on serum AMH concentration, her body weight. The dose is based on a recent determination of AMH (i.e. within the last 12 months), for example measured by the ELECSYS® AMH Plus immunoassay (Roche), or similar assays such as ACCESS AMH Advanced from Beckman Coulter or UMIPULSE G AMH from Fujirebio.

The term “follicle” herein means an ovarian follicle which is a fluid-filled sac that contains an immature egg, or oocyte.

A blastocyst forms in the early development of a human (or other mammal). In humans, blastocyst formation begins about 5 days after fertilization. The use of blastocysts in (IVF) generally involves retrieval (harvesting) from the woman a number of oocytes resulting from a controlled ovarian stimulation cycle; fertilization (insemination of) one or more oocytes and culturing the fertilized egg (oocyte) for five days to form a blastocyst (i.e. allowing the fertilized oocyte to develop to the blastocyst stage); and implanting the blastocyst into the uterus.

An embryo forms in the early development of a human (or other mammal). The use of embryos in (IVF) generally involves retrieval (harvesting) from the woman a number of oocytes resulting from a controlled ovarian stimulation cycle; fertilization (insemination of) one or more oocytes and culturing the fertilized egg (oocyte) for e.g. 3 days to form an embryo (i.e. allowing the fertilized oocyte to develop to the embryo stage); and implanting the embryo into the uterus.

In accordance with all aspects described herein, it is preferred that the treatment of infertility described herein, is or includes, a step of COS. The cause of infertility could be the woman's partner suffering from male infertility, although it will be appreciated that according to the present invention it is the woman (female) who is treated by COS.

A treatment of infertility as described herein may be for, and may be effective for, development of multiple follicles and pregnancy after fresh and/or cryopreserved embryo transfer in ovulatory women undergoing assisted reproductive technology (ART).

A treatment of infertility as described herein may be for, and may be effective for, promoting good quality blastocysts (e.g., category 3BB or higher blastocysts, e.g., treatment of infertility to increase the number of category 3BB or higher blastocysts on day 5 after oocyte retrieval) and/or to improve embryo implantation. The treatment of infertility may be treatment of infertility to increase the number of category 3BB or higher blastocysts on day 5 after oocyte retrieval (e.g., as compared to treatment with GONAL-F®). The treatment of infertility may be treatment of infertility to increase the number of fertilised (2PN) oocytes (e.g., as compared to treatment with GONAL-F®).

As used herein, “day one of treatment”, also referred to as “day one of stimulation”, refers to the first day that the dose of (e.g., recombinant) FSH is administered to the patient. Day one of treatment (stimulation) may take place on day 1, 2 or 3, for example on day 2 or day 3, of the patient's menstrual cycle. In other words, day one of treatment (stimulation) may be one, two or three days, for example two or three days, after the patient commences menstrual bleeding, consistent with usage of this term in clinical practice with GnRH antagonist or GnRH agonist protocols. The term “during treatment” means on a day or on days that FSH is being administered to the patient.

In the treatments, methods and uses described herein, the administration of recombinant FSH starts on day one of treatment and may continue for two to twenty days, for example continue for 2, 3, 4, 5, 6, 7, 8, 9, 10, 11, 12, 13, 14, 15, 16, 17, 18, 19, or 20 days. The dose administered on day 1 is referred to herein as the “starting dose”. The administration of recombinant FSH starts on day one of treatment and may continue for four to twenty days, for example seven to thirteen days, for example nine to thirteen days, for example 10 to 13 days, for example 10 to 11 days. The dose may be the same every day. However, variation of the dose depending on the patient's ovarian response (e.g., as measured by ultrasonography) is more likely.

In accordance with all aspects described herein, the recombinant FSH may be human cell line-derived recombinant FSH as described in more detail below. In all aspects, the recombinant FSH may be that sold under the trademark REKOVELLE® (follitropin delta) (Ferring B.V.). In all aspects, the recombinant FSH may be administered by injection, e.g., subcutaneous injection.

In accordance with all aspects described herein, the recombinant FSH composition (e.g., pharmaceutical composition) or medicament may be administered after pre-treatment of the patient with a (different) pharmaceutical composition, herein termed “composition A”, which suppresses endogenous gonadotropin production prior to day one of the treatment with rFSH. In other words, the composition (e.g., pharmaceutical composition) or medicament may be administered after the subject has been (pre-) treated with composition A, wherein composition A is a steroid, a GnRH agonist, a GnRH antagonist, etc. Herein, the term “pre-treated” or “pre-treatment” refers to administration of the pharmaceutical composition which suppresses endogenous gonadotropin production prior to day one of the treatment with rFSH (i.e., prior to day 1 of treatment), consistent with usage of this term in clinical practice with long GnRH agonist protocols.

Thus, the composition (e.g., pharmaceutical composition) or medicament for use described herein may be for administration 12 to 16, e.g., 13 to 15, e.g., 14 days, after administration of (e.g., after initiation of administration of, e.g., after initiation of daily administration of) a GnRH agonist (e.g., SYNAREL®, LUPRON®, DECAPEPTYL®). Additionally or alternatively, the recombinant FSH composition for use described herein may be for administration with a GnRH agonist.

Alternatively, the recombinant FSH composition (e.g., pharmaceutical composition) or medicament may be administered, or may be for administration, prior to administration of a GnRH antagonist (e.g., GANIRELIX®, CETRORELIX®), for example for administration five or six days prior to administration of a GnRH antagonist (i.e., for administration such that day 1 of stimulation is 5 or 6 days prior to administration of a GnRH antagonist). Additionally or alternatively, the recombinant FSH composition (e.g., pharmaceutical composition) for use described herein may be for administration with a GnRH antagonist.

Typically, in accordance with all aspects described herein, the recombinant FSH composition (e.g., pharmaceutical composition) or medicament is administered, or is for administration, prior to administration of a high (ovulatory) dose of human chorionic gonadotropin (hCG) (for example 4,000 to 11,000 IU hCG, e.g., 5,000 IU hCG, 10,000 IU hCG, etc.; or 150 to 500 μg recombinant hCG, for example 250 μg recombinant hCG); to induce final follicular maturation. Thus, in some embodiments, the methods described herein further comprise administration of a high (ovulatory) dose of human chorionic gonadotropin (hCG).

In accordance with all aspects described herein, the treatment (of infertility) and uses described herein may further comprise: retrieving (e.g., harvesting) oocyte(s); fertilizing (e.g., inseminating) the oocytes (s); and allowing the fertilized oocytes to develop to the embryo/blastocyst stage. The fertilization (e.g., insemination) may be in vitro fertilization, optionally intra-cytoplasmic sperm injection (ICSI).

In accordance with all aspects described herein, the treatment (of infertility) and uses described herein may further comprise assessing the quality of embryo/blastocysts obtained after fertilization of the harvested oocytes [e.g., to identify one or more good quality (i.e. grade 3BB or above) blastocysts]. Assessment of blastocyst quality may take place on day 5 after oocyte retrieval and may study three parameters: blastocyst expansion and hatching status (grade 1-6), blastocyst inner cell mass grading (grade A-D) and trophectoderm grading (grade A-D), as is well known in the art. Blastocysts can be given a numerical score by using the system of Gardner & Schoolcraft, as is well known in the art, with the addition of D-categories for inner cell mass and trophectoderm.

Early pregnancy loss is generally defined as a pregnancy loss occurring before ongoing pregnancy. Herein, the term “early pregnancy loss” is defined as a pregnancy loss occurring between a positive pregnancy test performed 13-15 days after embryo/blastocyst transfer and a subsequent assessment of pregnancy (e.g. by transvaginal ultrasound) 10-11 weeks after embryo/blastocyst transfer.

In accordance with all aspects described herein, the treatment of infertility and uses described herein may further comprise transfer of one or more embryo/blastocyst(s) identified by assessment of quality of the embryo/blastocysts (e.g., fresh embryo/blastocyst transfer). In specific embodiments, a single embryo/blastocyst is transferred. In specific embodiments, a single embryo is, or two embryos are, transferred.

In accordance with all aspects described herein, the treatment (of infertility) and uses described herein may further comprise freezing one or more embryo/blastocysts identified by assessment of quality of the embryo/blastocysts (for later transfer).

Thus, In accordance with all aspects described herein, the treatment (of infertility) and uses described herein may further comprise—in addition to optional administration of a GnRH agonist or antagonist, administration of recombinant FSH, and administration of an ovulatory dose of hCG, retrieving (e.g., harvesting) oocyte(s); fertilizing (e.g., inseminating) the oocyte(s) —allowing the fertilized oocytes to develop to the embryo/blastocyst stage and cryopreserving one or more embryo/blastocysts (e.g., embryo/blastocysts identified by assessment of quality of the embryo/blastocysts, e.g., for later transfer).

The compositions, uses and methods described herein may be for, and may be effective for, reducing early pregnancy loss and/or increase the probability of live birth in a patient treated for infertility.

The recombinant FSH doses listed herein may be for treatment of infertility in the patient's (subject's) first stimulation protocol (first stimulation “cycle”) by the methods and treatment protocols described herein. Thus, the composition(s) may be for use in the treatment of infertility in a patient (subject) who has not previously been treated for infertility by controlled ovarian stimulation; for use in the treatment of infertility in a patient (subject) who has not previously completed a treatment for infertility by controlled ovarian stimulation; or for use in the treatment of infertility in a patient (subject) who has not been treated for infertility by controlled ovarian stimulation in the previous six months, more preferably a patient (subject) who has not been treated for infertility by controlled ovarian stimulation in the previous twelve months. It will be appreciated that for further stimulation cycles (that is, treatments of infertility by controlled ovarian stimulation) by the methods and treatment protocols described herein, the doses may be adjusted according to actual ovarian response in the first cycle by the methods and treatment protocols described herein.

Treatments

In a trial comparing REKOVELLE® with a CHO cell derived rFSH, the applicants unexpectedly found that early pregnancy loss was reported for 20% of subjects with a positive βhCG test treated with REKOVELLE®, compared to 34.6% of subjects with a positive βhCG test treated with the CHO cell derived rFSH (Example 1). Similarly, the Examples have shown that REKOVELLE® treatment increases the probability of a live birth, compared to treatment with a CHO cell derived rFSH. In a first aspect, there is provided a composition (e.g., a pharmaceutical composition) comprising recombinant follicle stimulating hormone (rFSH) for use in reducing the likelihood of early pregnancy loss in a patient treated for infertility by controlled ovarian stimulation, wherein the recombinant FSH includes α2,3- and α2,6-sialylation (e.g. wherein 1% to 60% of the total sialylation is α2,6-sialylation and wherein 40% to 99% of the total sialylation is α2,3-sialylation, e.g. wherein 5% to 20% of the total sialylation is α2,6-sialylation and wherein 80% to 95% of the total sialylation is α2,3-sialylation). For example, the composition may be for administration to a patient having serum AMH level of <15 pmol/L, wherein the composition is to be administered at a dose of, or equivalent to, 11 to 13 μg recombinant FSH per day. This use may comprise a step of determining the serum AMH level of the patient (e.g. using the automated Elecsys® AMH assay from Roche Diagnostics or similar), and a step of administering the dose to a patient having serum AMH level of <15 pmol/L. In another example, the composition may be for administration to a patient having a serum AMH level of ≥15 pmol/L, wherein the composition is to be administered at a dose of, or equivalent to, 0.09 to 0.19 μg recombinant FSH per kg bodyweight of the patient per day. This use may comprise a step of determining the serum AMH level of the patient, and a step of administering the dose to a patient having serum AMH level of ≥15 pmol/L. In some examples, the patient may be (for example is identified as being) a female of Japanese ethnicity, for example a female with two ethnic Japanese parents. This use may further comprise a step of identifying a patient of Japanese ethnicity (e.g. prior to treatment). In some examples, the patient may be (for example is identified as being) a female of Asian (e.g. Chinese, Taiwanese, Vietnamese or South Korean) ethnicity, for example a female with two ethnic Asian (e.g. Chinese, Taiwanese, Vietnamese or South Korean) parents. This use may further comprise a step of identifying a patient of Asian (e.g. Chinese, Taiwanese, Vietnamese or South Korean) ethnicity (e.g. prior to treatment). The skilled person will readily understand how to collect information on ethnicity (e.g. visually and/or by questionnaire) to identify a patent as being of Japanese or Asian (e.g. Chinese, Taiwanese, Vietnamese or South Korean) ethnicity prior to treatment.

The composition (and method) may be for use in patients/subjects who are predicted as (or identified as) high responders. As used herein, subjects classified as being “predicted to have a high ovarian response to controlled ovarian stimulation” or as a “predicted high responder” refers to women who are likely to develop high numbers of follicles or oocytes following a standard protocol of controlled ovarian stimulation (COS), such as women with a greater than average likelihood of producing 15 or more oocytes. Women may be identified as being predicted high responders if they have generated 15 or more oocytes in a previous ART cycle, e.g., in a previous COS treatment. Additionally or alternatively, women may be identified as being predicted high responders if they are considered to be at risk of developing OHSS. Additionally or alternatively, women may be identified as being predicted high responders if they have a serum level of anti-Mullerian hormone (AMH) 15 pmol/L, such as a serum AMH level 35.7±0.5 pmol/L (5.0±0.2 ng/ml), when measured using a Beckmann-Coulter Gen 2 assay as described in Arce et al., Fertility and Sterility 99: 1644-53 (2013), or an equivalent AMH level assessed by a different method. The use/method may include a step of identifying a patient as a high responder prior to treatment. It is believed that treatment of high responders with FSH including 2,3- and 2,6-sialylation (such as Rekovelle®) is less likely to lead to cancellation of a COS cycle due to over-response, and may increase live birth rate.

In a further aspect, there is provided a composition (e.g., a pharmaceutical composition) comprising recombinant follicle stimulating hormone (FSH) for use in increasing the probability of live birth following treatment for infertility by controlled ovarian stimulation, wherein the recombinant FSH includes α2,3- and α2,6-sialylation (e.g. wherein 1% to 60% of the total sialylation is α2,6-sialylation and wherein 40% to 99% of the total sialylation is α2,3-sialylation, e.g. wherein 5% to 20% of the total sialylation is α2,6-sialylation and wherein 80% to 95% of the total sialylation is α2,3-sialylation). For example, the composition may be for administration to a patient having serum AMH level of <15 pmol/L, wherein the composition is to be administered at a dose of, or equivalent to, 11 to 13 μg recombinant FSH per day. This use may comprise a step of determining the serum AMH level of the patient, and a step of administering the dose to a patient having serum AMH level of <15 pmol/L. In another example, the composition may be for administration to a patient having a serum AMH level of ≥15 pmol/L, wherein the composition is to be administered at a dose of, or equivalent to, 0.09 to 0.19 μg recombinant FSH per kg bodyweight of the patient per day. The minimum dose may be 6 μg recombinant FSH per day. This use may comprise a step of determining the serum AMH level of the patient, and a step of administering the dose to a patient having serum AMH level of ≥15 pmol/L. In some examples, the patient may be (for example is identified as being) a female of Japanese ethnicity, for example a female with two ethnic Japanese parents. This use may further comprise a step of identifying a patient of Japanese ethnicity (e.g. prior to treatment). In some examples, the patient may be (for example is identified as being) a female of Asian (e.g. Chinese, Taiwanese, Vietnamese or South Korean) ethnicity, for example a female with two ethnic Asian (e.g. Chinese, Taiwanese, Vietnamese or South Korean) parents. This use may further comprise a step of identifying a patient of Asian (e.g. Chinese, Taiwanese, Vietnamese or South Korean) ethnicity (e.g. prior to treatment). The skilled person will readily understand how to collect information on ethnicity (e.g. visually and/or by questionnaire) to identify a patient as being of Japanese or Asian (e.g. Chinese, Taiwanese, Vietnamese or South Korean) ethnicity prior to treatment.

The composition (and method) may be for use in patients/subjects who are predicted as (or identified as) high responders. As used herein, subjects classified as being “predicted to have a high ovarian response to controlled ovarian stimulation” or as a “predicted high responder” refers to women who are likely to develop high numbers of follicles or oocytes following a standard protocol of controlled ovarian stimulation (COS), such as women with a greater than average likelihood of producing 15 or more oocytes. Women may be identified as being predicted high responders if they have generated 15 or more oocytes in a previous ART cycle, e.g., in a previous COS treatment. Additionally or alternatively, women may be identified as being predicted high responders if they are considered to be at risk of developing OHSS. Additionally or alternatively, women may be identified as being predicted high responders if they have a serum level of anti-Mullerian hormone (AMH)≥15 pmol/L, such as a serum AMH level ≥35.7±0.5 pmol/L (≥5.0±0.2 ng/ml), when measured using a Beckmann-Coulter Gen 2 assay as described in Arce et al., Fertility and Sterility 99: 1644-53 (2013), or an equivalent AMH level assessed by a different method. The use/method may include a step of identifying a patient as a high responder prior to treatment. It is believed that treatment of high responders with FSH including 2,3- and 2,6-sialylation (such as Rekovelle®) is less likely to lead to cancellation of a COS cycle due to over-response, and may increase live birth rate.

In a further aspect, there is provided a composition (e.g. a pharmaceutical composition) comprising recombinant follicle stimulating hormone (FSH) for use in the treatment of infertility (e.g. by controlled ovarian stimulation) in a female patient of age 35 years or greater, for example of age 36 years or greater, for example of age 37 years or greater, for example of age 38 years or greater, for example a female patient of age 38-40 years, to increase the probability of live birth, wherein the recombinant FSH includes α2,3- and α2,6-sialylation (e.g. wherein 1% to 60% of the total sialylation is α2,6-sialylation and wherein 40% to 99% of the total sialylation is α2,3-sialylation, e.g. wherein 5% to 20% of the total sialylation is α2,6-sialylation and wherein 80% to 95% of the total sialylation is α2,3-sialylation). The treatment may comprise a step of determining the age of the patient, and a step of administering the recombinant FSH to a patient of age 35 years or greater, for example of age 36 years or greater, for example of age 37 years or greater, for example of age 38 years or greater, for example a female patient of age 38-40 years (to increase thereby the probability of live birth). The skilled person will readily appreciate how to determine the age of a patient, e.g. by request, questionnaire etc. Preferably the patient is of, or is determined to be of, age 38 years or greater, for example a female patient of, or is determined to be of, age 38-40 years. For example, the composition may be for administration to a patient having serum AMH level of <15 pmol/L, wherein the composition is to be administered at a dose of, or equivalent to, 11 to 13 μg recombinant FSH per day. This use may comprise a step of determining the serum AMH level of the patient, and a step of administering the dose to a patient having serum AMH level of <15 pmol/L. In another example, the composition may be for administration to a patient having a serum AMH level of ≥15 pmol/L, wherein the composition is to be administered at a dose of, or equivalent to, 0.09 to 0.19 μg recombinant FSH per kg bodyweight of the patient per day. The minimum dose may be 6 μg recombinant FSH per day. This use may comprise a step of determining the serum AMH level of the patient, and a step of administering the dose to a patient having serum AMH level of ≥15 pmol/L. In some examples, the patient may be (for example is identified as being) a female of Japanese ethnicity, for example a female with two ethnic Japanese parents. This use may further comprise a step of identifying a patient of Japanese ethnicity (e.g. prior to treatment). In some examples, the patient may be (for example is identified as being) a female of Asian (e.g. Chinese, Taiwanese, Vietnamese or South Korean) ethnicity, for example a female with two ethnic Asian (e.g. Chinese, Taiwanese, Vietnamese or South Korean) parents. This use may further comprise a step of identifying a patient of Asian (e.g. Chinese, Taiwanese, Vietnamese or South Korean) ethnicity (e.g. prior to treatment). The skilled person will readily understand how to collect information on ethnicity (e.g. visually and/or by questionnaire) to identify a patient as being of Japanese or Asian (e.g. Chinese, Taiwanese, Vietnamese or South Korean) ethnicity prior to treatment.

The composition (and method) may be for use in patients/subjects who are predicted as (or identified as) high responders. As used herein, subjects classified as being “predicted to have a high ovarian response to controlled ovarian stimulation” or as a “predicted high responder” refers to women who are likely to develop high numbers of follicles or oocytes following a standard protocol of controlled ovarian stimulation (COS), such as women with a greater than average likelihood of producing 15 or more oocytes. Women may be identified as being predicted high responders if they have generated 15 or more oocytes in a previous ART cycle, e.g., in a previous COS treatment. Additionally or alternatively, women may be identified as being predicted high responders if they are considered to be at risk of developing OHSS. Additionally or alternatively, women may be identified as being predicted high responders if they have a serum level of anti-Müllerian hormone (AMH)≥15 pmol/L, such as a serum AMH level ≥35.7±0.5 pmol/L (≥5.0±0.2 ng/ml), when measured using a Beckmann-Coulter Gen 2 assay as described in Arce et al., Fertility and Sterility 99: 1644-53 (2013), or an equivalent AMH level assessed by a different method. The use/method may include a step of identifying a patient as a high responder prior to treatment. It is believed that treatment of high responders with FSH including 2,3- and 2,6-sialylation (such as Rekovelle®) is less likely to lead to cancellation of a COS cycle due to over-response, and may increase live birth rate.

In a further aspect, there is provided a composition (e.g. a pharmaceutical composition) comprising recombinant follicle stimulating hormone (FSH) for use in the treatment of infertility (e.g. by controlled ovarian stimulation) in a female patient of age 28 years or greater, for example of age 30 years or greater, for example of age 31 years or greater, for example of age 32 years or greater, for example of age 33 years or greater, for example of age 35 years or greater, for example of age 36 years or greater, for example of age 37 years or greater, for example of age 38 years or greater, for example a female patient of age 28-40 years, for example a female patient of age 30-40 years, to increase the probability of live birth, wherein the recombinant FSH includes α2,3- and α2,6-sialylation (e.g. wherein 1% to 60% of the total sialylation is α2,6-sialylation and wherein 40% to 99% of the total sialylation is α2,3-sialylation, e.g. wherein 5% to 20% of the total sialylation is α2,6-sialylation and wherein 80% to 95% of the total sialylation is α2,3-sialylation). The treatment may comprise a step of determining the age of the patient, and a step of administering the recombinant FSH to a patient of age 28 years or greater, for example of age 30 years or greater, for example a female patient of age 28-40 years, for example a female patient of age 30-40 years (to increase thereby the probability of live birth). The skilled person will readily appreciate how to determine the age of a patient, e.g. by request, questionnaire etc. Preferably the patient is of, or is determined to be of, age 28 years or greater, for example a female patient of, or is determined to be of, age 28-40 years. More preferably the patient is of, or is determined to be of, age 30 years or greater, for example a female patient of, or is determined to be of, age 30-40 years. For example, the composition may be for administration to a patient having serum AMH level of <15 pmol/L, wherein the composition is to be administered at a dose of, or equivalent to, 11 to 13 μg recombinant FSH per day. This use may comprise a step of determining the serum AMH level of the patient, and a step of administering the dose to a patient having serum AMH level of <15 pmol/L. In another example, the composition may be for administration to a patient having a serum AMH level of ≥15 pmol/L, wherein the composition is to be administered at a dose of, or equivalent to, 0.09 to 0.19 μg recombinant FSH per kg bodyweight of the patient per day. The minimum dose may be 6 μg recombinant FSH per day. This use may comprise a step of determining the serum AMH level of the patient, and a step of administering the dose to a patient having serum AMH level of ≥15 pmol/L. In some examples, the patient may be (for example is identified as being) a female of Japanese ethnicity, for example a female with two ethnic Japanese parents. This use may further comprise a step of identifying a patient of Japanese ethnicity (e.g. prior to treatment). In some examples, the patient may be (for example is identified as being) a female of Asian (e.g. Chinese, Taiwanese, Vietnamese or South Korean) ethnicity, for example a female with two ethnic Asian (e.g. Chinese, Taiwanese, Vietnamese or South Korean) parents. This use may further comprise a step of identifying a patient of Asian (e.g. Chinese, Taiwanese, Vietnamese or South Korean) ethnicity (e.g. prior to treatment). The skilled person will readily understand how to collect information on ethnicity (e.g. visually and/or by questionnaire) to identify a patient as being of Japanese or Asian (e.g. Chinese, Taiwanese, Vietnamese or South Korean) ethnicity prior to treatment.

In a further aspect, there is provided a composition (e.g. a pharmaceutical composition) comprising recombinant follicle stimulating hormone (FSH) for use in the treatment of infertility (e.g. by controlled ovarian stimulation) in a female patient of age 30 to 40 years, to increase the probability of live birth, wherein the recombinant FSH includes α2,3- and α2,6-sialylation. The treatment may comprise a step of determining the age of the patient, and a step of administering the recombinant FSH to a patient of age 30-40 years. In a further aspect, there is provided a composition (e.g. a pharmaceutical composition) comprising recombinant follicle stimulating hormone (FSH) for use in the treatment of infertility (e.g. by controlled ovarian stimulation) in a female patient of age 30 to 37 years, to increase the probability of live birth, wherein the recombinant FSH includes α2,3- and α2,6-sialylation. The treatment may comprise a step of determining the age of the patient, and a step of administering the recombinant FSH to a patient of age 30-37 years. In a further aspect, there is provided a composition (e.g. a pharmaceutical composition) comprising recombinant follicle stimulating hormone (FSH) for use in the treatment of infertility (e.g. by controlled ovarian stimulation) in a female patient of age 36 to 40 years, to increase the probability of live birth, wherein the recombinant FSH includes α2,3- and α2,6-sialylation. The treatment may comprise a step of determining the age of the patient, and a step of administering the recombinant FSH to a patient of age 36-40 years. For example, the patient may have serum AMH level of <15 pmol/L, wherein the composition is to be administered at a dose of, or equivalent to, 11 to 13 μg recombinant FSH per day. In this example, the use may comprise a step of determining the serum AMH level of the patient, and a step of administering the dose to a patient having serum AMH level of <15 pmol/L. In another example, the patient may have serum AMH level of ≥15 pmol/L, wherein the composition is to be administered at a dose of, or equivalent to, 0.09 to 0.19 μg recombinant FSH per kg bodyweight of the patient per day. In this example, the use may comprise a step of determining the serum AMH level of the patient, and a step of administering the dose to a patient having serum AMH level of ≥15 pmol/L. In a further aspect, there is provided a composition (e.g. a pharmaceutical composition) comprising recombinant follicle stimulating hormone (FSH) for use in the treatment of infertility (e.g. by controlled ovarian stimulation) in a female patient of age 35 to 40 years, to increase the probability of live birth, wherein the recombinant FSH includes α2,3- and α2,6-sialylation, wherein if the patient has serum AMH level of <15 pmol/L, the composition is to be administered at a dose of, or equivalent to, 11 to 13 μg recombinant FSH per day, and if the patient has serum AMH level of ≥15 pmol/L, the composition is to be administered at a dose of, or equivalent to, 0.09 to 0.19 μg recombinant FSH per kg bodyweight of the patient per day. In this aspect, the use may comprise a step of determining the age of the patient, and a step of administering the recombinant FSH to a patient of age 35-40 years. The use may comprise a step of determining the serum AMH level of the patient, and a step of administering the specified dose to a patient having the specified serum AMH level. In some examples, the patient may be (for example is identified as being) a female of Japanese ethnicity, for example a female with two ethnic Japanese parents. This use may further comprise a step of identifying a patient of Japanese ethnicity (e.g. prior to treatment). In some examples, the patient may be (for example is identified as being) a female of Asian (e.g. Chinese, Taiwanese, Vietnamese or South Korean) ethnicity, for example a female with two ethnic Asian (e.g. Chinese, Taiwanese, Vietnamese or South Korean) parents. This use may further comprise a step of identifying a patient of Asian (e.g. Chinese, Taiwanese, Vietnamese or South Korean) ethnicity (e.g. prior to treatment). The skilled person will readily understand how to collect information on ethnicity (e.g. visually and/or by questionnaire) to identify a patient as being of Japanese or Asian (e.g. Chinese, Taiwanese, Vietnamese or South Korean) ethnicity prior to treatment. In a further aspect, there is provided a composition (e.g. a pharmaceutical composition) comprising recombinant follicle stimulating hormone (FSH) for use in the treatment of infertility (e.g. by controlled ovarian stimulation) in a female patient of age 35 years or greater, for example of age 36 years or greater, to increase the probability of live birth, wherein the recombinant FSH includes α2,3- and α2,6-sialylation, and wherein the patient is of Japanese or Asian ethnicity. The use may comprise a step of determining the age of the patient, and a step of administering the recombinant FSH to a patient of age 35 years or greater, for example of age 36 years or greater. In aspects, the recombinant FSH may be a recombinant FSH wherein 1% to 60% of the total sialylation of the FSH is α2,6-sialylation and wherein 40% to 99% of the total sialylation of the FSH is α2,3-sialylation, e.g. wherein 5% to 20% of the total sialylation is α2,6-sialylation and wherein 80% to 95% of the total sialylation is α2,3-sialylation). Thus, the recombinant FSH may include α2,3- and α2,6-sialylation wherein 5% to 20% of the total sialylation is α2,6-sialylation and wherein 80% to 95% of the total sialylation is α2,3-sialylation. The recombinant FSH may be a recombinant FSH which has been produced or expressed in a human cell line.

The composition (and method) may be for use in patients/subjects who are predicted as (or identified as) high responders. As used herein, subjects classified as being “predicted to have a high ovarian response to controlled ovarian stimulation” or as a “predicted high responder” refers to women who are likely to develop high numbers of follicles or oocytes following a standard protocol of controlled ovarian stimulation (COS), such as women with a greater than average likelihood of producing 15 or more oocytes. Women may be identified as being predicted high responders if they have generated 15 or more oocytes in a previous ART cycle, e.g., in a previous COS treatment. Additionally or alternatively, women may be identified as being predicted high responders if they are considered to be at risk of developing OHSS. Additionally or alternatively, women may be identified as being predicted high responders if they have a serum level of anti-Mullerian hormone (AMH) 15 pmol/L, such as a serum AMH level 35.7±0.5 pmol/L (5.0±0.2 ng/ml), when measured using a Beckmann-Coulter Gen 2 assay as described in Arce et al., Fertility and Sterility 99: 1644-53 (2013), or an equivalent AMH level assessed by a different method. The use/method may include a step of identifying a patient as a high responder prior to treatment. It is believed that treatment of high responders with FSH including 2,3- and 2,6-sialylation (such as Rekovelle®) is less likely to lead to cancellation of a COS cycle due to over-response, and may increase live birth rate.

Also provided are methods of reducing the likelihood of early pregnancy loss in a patient treated for infertility by controlled ovarian stimulation, the methods comprising administering to the patient a pharmaceutically effective amount of a composition comprising recombinant FSH; wherein the recombinant FSH includes α2,3- and α2,6-sialylation (e.g. wherein 1% to 60% of the total sialylation is α2,6-sialylation and wherein 40% to 99% of the total sialylation is α2,3-sialylation, e.g. wherein 5% to 20% of the total sialylation is α2,6-sialylation and wherein 80% to 95% of the total sialylation is α2,3-sialylation), and methods of increasing the probability of live birth following treatment of a patient for infertility by controlled ovarian stimulation, the methods comprising administering to the patient a pharmaceutically effective amount of a composition comprising recombinant FSH; wherein the recombinant FSH includes α2,3- and α2,6-sialylation (e.g. wherein 5% to 20% of the total sialylation is α2,6-sialylation and wherein 80% to 95% of the total sialylation is α2,3-sialylation).

As noted above, the administration of recombinant FSH starts on day one of treatment and may continue for two to twenty days. The dose may be the same every day. However, variation of the dose depending on the patient's ovarian response (e.g., as measured by ultrasonography) is more likely.

As noted above, the recombinant FSH composition (e.g., pharmaceutical composition) or medicament may be administered after pre-treatment of the patient with a (different) pharmaceutical composition, herein termed “composition A”, which suppresses endogenous gonadotropin production prior to day one of the treatment with rFSH, such as a steroid, a GnRH agonist, a GnRH antagonist etc.

As noted above, typically, the recombinant FSH composition (e.g., pharmaceutical composition) or medicament is administered, or is for administration, prior to administration of a high (ovulatory) dose of human chorionic gonadotropin (hCG) (for example, 4,000 to 11,000 IU hCG, e.g., 5,000 IU hCG, 10,000 IU hCG, etc.; or 150 to 500 μg recombinant hCG, for example 250 μg recombinant hCG); to induce final follicular maturation. In some embodiments, therefore, the methods described herein further comprise administration of a high (ovulatory) dose of human chorionic gonadotropin (hCG).

As noted above, the treatment (of infertility) and uses may further comprise: retrieving (e.g., harvesting) oocyte(s); fertilizing (e.g., inseminating) the oocytes (s); and allowing the fertilized oocytes to develop to the embryo/blastocyst stage. As noted above, the treatment of infertility may further comprise assessing the quality of embryo/blastocysts and fresh transfer of embryo/blastocyst(s) or freezing of embryo/blastocysts for later transfer.

As with the first aspect discussed above, treatment in accordance with this aspect may further comprise administration of a high (ovulatory) dose of human chorionic gonadotropin (hCG), and, optionally, retrieving (e.g., harvesting) oocyte(s); fertilizing (e.g., inseminating) the oocytes (s); allowing the fertilized oocytes to develop to the embryo/blastocyst stage, further optionally assessing the quality of embryo/blastocysts and fresh transfer of embryo/blastocyst(s) or freezing of embryo/blastocysts for later transfer. Additionally or alternatively, the treatment may further comprise monitoring and/or control of over-response to treatment (e.g., OHSS).

Recombinant FSH and rFSH Compositions

As noted above, the methods and compositions described herein use recombinant FSH (rFSH). FSH comprises a 92 amino acid alpha sub-unit, also common to the other glycoprotein hormones LH and chorionic gonadotropin (CG), and a 111 amino acid beta sub-unit unique to FSH that confers the biological specificity of the hormone (Pierce and Parsons, 1981). Each sub-unit is post translationally modified by the addition of complex carbohydrate residues. Both subunits carry 2 sites for N-linked glycan attachment, the alpha sub-unit at amino acids 52 and 78 and the beta sub-unit at amino acid residues 7 and 24 (Rathnam and Saxena, 1975, Saxena and Rathnam, 1976). FSH is thus glycosylated to about 30% by mass (Dias and Van Roey. 2001. Fox et al. 2001).

The glycosylation of rFSH products reflects the range of glycosyl-transferases present in the host cell line. Commercially available rFSH products derived from engineered CHO cells have a more limited range of glycan modifications than those found on the natural products. Examples of the reduced glycan heterogeneity found in CHO cell derived rFSH include a lack of bisecting glucosamine and a reduced content of core fucosylation and acetyl lactosamine extensions (Hard et al., 1990). In addition, CHO cells are only able to add sialic acid using the α2,3 linkage (Kagawa et al, 1988, Takeuchi et al, 1988, Svensson et al., 1990); CHO cell-derived rFSH only includes α2,3-linked sialic acid and does not include α2,6-linked sialic acid. Thus, CHO cell-derived rFSH is different from naturally produced FSH (e.g., human pituitary/serum/urinary FSH) which contains glycans with a mixture of α2,3 and α2,6-linked sialic acid, with a predominance of the former.

As noted above, the present applicants have developed a human cell line-derived rFSH which is the subject of International Patent Application No. PCT/GB2009/000978, published as WO2009/127826A, and also approved by the EC as REVOKELLE® (follitropin delta, also known as FE 999049). Recombinant FSH with a mixture of both α2,3 and α2,6-linked sialic acid was made by engineering a human cell line to express both rFSH and α2,3 sialyltransferase. The amino acid sequence of the human cell line-derived recombinant FSH which is the subject of International Patent Application No. PCT/GB2009/000978, published as WO2009/127826A (e.g., FE 999049), is the native human FSH sequence, but the product has a different glycosylation pattern. The expressed product is highly acidic and carries a mix of both α2,3- and α2,6-linked sialic acids; the latter provided by the endogenous sialyl transferase activity. It was found that the type of sialic acid linkage, α2,3- or α2,6-, can have a dramatic influence on biological clearance of FSH. Thus REVOKELLE® (e.g., FE 999049) may be more biologically appropriate compared to CHO cell-derived recombinant products that have only α2,3 linked sialic acid (Kagawa et al, 1988, Takeuchi et al, 1988, Svensson et al., 1990) and have decreased sialic acid content (Ulloa-Aguirre et al. 1995, Andersen et al. 2004).

Thus, the recombinant FSH used in accordance with the methods and compositions described herein may be produced or expressed in a human cell line, such as a PER.C6® cell line. The recombinant FSH may be produced or expressed in a PER.C6® cell line, a PER.C6® derived cell line or a modified PER.C6® cell line. Recombinant FSH which is produced or expressed in a PER.C6® cell line will include some α2,6-linked sialic acids (α2,6 sialylation) provided by endogenous sialyl transferase activity (of the cell line) and will include some α2,3-linked sialic acids (α2,3 sialylation) provided by endogenous sialyl transferase activity. The cell line may be modified using α2,3-sialyltransferase. The cell line may be modified using α2,6-sialyltransferase. Alternatively or additionally, the recombinant FSH may include α2,6-linked sialic acids (α2,6 sialylation) provided by endogenous sialyl transferase activity (of the cell line). Herein, the term “human-derived recombinant FSH” means recombinant FSH which is produced or expressed in a human cell line (e.g., recombinant FSH made by engineering a human cell line).

The recombinant FSH used in the methods and compositions described herein may include α2,3- and α2,6-sialylation. The recombinant FSH for use according to the invention may have 1% to 99% of the total sialylation being α2,3-sialylation. The recombinant FSH for use according to the invention may have 1% to 99% of the total sialylation being α2,6-sialylation. The recombinant FSH may have 1% to 50% of the total sialylation as α2, 6-sialyation, and 50% to 99% of the total sialylation as 2,3-sialyation. For example, 80% to 95%, for example 80% to 90%, for example 82% to 89%, for example 85% to 89% of the total sialylation may be α2,3-sialylation. For example, 5% to 20%, for example 10% to 20%, for example 11% to 18%, for example 11% to 15%, of the total sialylation may be α2,6-sialylation. In an example, the recombinant FSH has 5% to 20% of the total sialylation as α2, 6-sialyation, and 80% to 95% of the total sialylation as 2,3-sialyation. In another example, the recombinant FSH has 50% to 80% of the total sialylation as α2, 6-sialyation, and 20% to 50% of the total sialylation as 2,3-sialyation.

Herein, by “sialylation”, it is meant the amount of sialic residues present on the recombinant FSH carbohydrate structures. Consistent with usage in the art, α2,3-sialylation means sialylation at the 2,3 position and α2,6 sialylation means sialylation at the 2,6 position. Thus “% of the total sialylation may be a 2,3 sialylation” refers to the % of the total number of sialic acid residues present in the FSH (or hCG) which are sialylated in the 2,3 position. The term “% of the total sialylation being α2,6-sialylation” refers to the % of the total number of sialic acid residues present in the FSH (or hCG) which are sialylated in the 2,6 position.

In all aspects, the rFSH may be present as a single isoform or as a mixture of isoforms.

The composition may be a pharmaceutical composition. The pharmaceutical composition is for the treatment of infertility. The treatment of infertility may comprise COS prior to ART. The pharmaceutical composition may be used, for example, in medical indications where known FSH preparations are used, in accordance with the methods and treatment protocols disclosed herein

The recombinant FSH, composition, or pharmaceutical composition can be formulated into well-known compositions for any route of drug administration, e.g., oral, rectal, parenteral, transdermal (e.g., patch technology), intravenous, intramuscular, subcutaneous (e.g., for subcutaneous injection), intracisternal, intravaginal, intraperitoneal, local (powders, ointments or drops) or as a buccal or nasal spray. A typical composition comprises a pharmaceutically acceptable carrier, such as aqueous solution, nontoxic excipients, including salts and preservatives, buffers and the like, as described in Remington's Pharmaceutical Sciences fifteenth edition (Matt Publishing Company, 1975), at pages 1405 to 1412 and 1461-87, and the national formulary XIV fourteenth edition (American Pharmaceutical Association, 1975), among others. For example, the recombinant FSH, composition or pharmaceutical composition can be formulated for injection, such as for subcutaneous injection.

Examples of suitable aqueous and non-aqueous pharmaceutical carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate.

The compositions of the present invention may also comprise additives such as but not limited to preservatives, wetting agents, emulsifying agents, surfactants and dispersing agents. Antibacterial and antifungal agents can be included to prevent growth of microbes and includes, for example, m-cresol, benzyl alcohol, paraben, chlorobutanol, phenol, sorbic acid, and the like. If a preservative is included, benzyl alcohol, phenol and/or m-cresol are preferred; however, the preservative is by no means limited to these examples. Furthermore, it may be desirable to include isotonic agents such as sugars, sodium chloride, amino acids and the like.

For example, the composition or medicament may comprise recombinant FSH and one or more of polysorbate 20, L-methionine, phenol, and arginine hydrochloride. Such a composition may be formulated for injection, such as for subcutaneous injection. For example, the composition or medicament may be the REKOVELLE® formulation (rFSH with excipients phenol, polysorbate 20, L-methionine, sodium sulphate decahydrate, disodium phosphate dodecahydrate, phosphoric acid [concentrated, for pH-adjustment], sodium hydroxide [for pH-adjustment], and water for injection).

Injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use. Injectable formulations can be supplied in any suitable container, e.g., vial, pre-filled syringe, injection cartridges, and the like.

The recombinant FSH, composition, or medicament may be formulated for single use or for multiple use (multiple dose). If the recombinant FSH, composition, or medicament is formulated for multiple use, typically one or more preservatives is included. If a preservative is included, benzyl alcohol, phenol or m-cresol, are preferred; however, the preservative is by no means limited to these examples. The single use or multiple use formulated composition or medicament may further comprise an amino acid or combination of amino acids. Typically, the amino acid is arginine, for example added as arginine or more typically arginine hydrochloride.

The recombinant FSH, composition, or medicament may be included in a container such as a vial, prefilled cartridge (e.g., for single administration or multiple use) or an injection device such as a “pen” for e.g., administration of multiple doses.

The recombinant FSH, composition or pharmaceutical composition may be a formulation (e.g., injectable formulation) including rFSH.

The recombinant FSH, composition or medicament can be supplied in any appropriate package. For example, a composition or medicament can include a number of containers (e.g., pre-filled syringes or vials) containing FSH. The syringes or vials may be packaged in a blister package or other means to maintain sterility. Any composition or medicament can optionally include instructions for using the FSH formulation.

The pH and exact concentration of the various components of the pharmaceutical composition are adjusted in accordance with routine practice in this field. See GOODMAN and GILMAN's THE PHARMACOLOGICAL BASIS FOR THERAPEUTICES, 7^{th} ed. In a typical embodiment, the recombinant FSH, composition or medicament are supplied as compositions for parenteral administration. General methods for the preparation of the parenteral formulations are known in the art and are described in REMINGTON; THE SCIENCE AND PRACTICE OF PHARMACY, supra, at pages 780-820. The parenteral compositions can be supplied in liquid formulation or as a solid which will be mixed with a sterile injectable medium just prior to administration. The parenteral compositions may be supplied in dosage unit form for ease of administration and uniformity of dosage.

In a further aspect there is provided the use of recombinant follicle stimulating hormone (FSH) in the manufacture of a medicament for the uses as described herein.

Further aspects are illustrated in the following examples, which are not limiting in any respect.

### EXAMPLES

The following examples use REKOVELLE®, follitropin delta, which is a recombinant FSH expressed in a PER.C6© cell line engineered by the methods disclosed in WO2013/020996 and WO2009/127826A.

The Marketing Authorisation holder for REKOVELLE® is Ferring Pharmaceuticals A/S of Kay Fiskers Plads 11, 2300 Copenhagen S, Denmark, and it is available in the UK from Ferring Pharmaceuticals of Drayton Hall, Church Road, West Drayton, UB7 7PS, UK.

The active substance in REKOVELLE® is follitropin delta (FE999049). REKOVELLE® is highly sialylated and includes α2,3- and α2,6-sialylation, with about 85% to 90% of the total sialylation being α2,3-sialylation and about 10% to 15% of the total sialylation being α2,6-sialylation.

REKOVELLE® is a clear and colourless solution for injection (injection). One millilitre of solution contains 33.3 micrograms of follitropin delta in each millilitre of solution. The other ingredients are phenol, polysorbate 20, L-methionine, sodium sulphate decahydrate, disodium phosphate dodecahydrate, concentrated phosphoric acid, sodium hydroxide and water for injections.

### Example 1: A Randomized, Controlled, Assessor-Blind, Multicentre Trial Assessing the Efficacy and Safety of FE 999049 (Rekovelle®) in Controlled Ovarian Stimulation in Japanese Women Undergoing an Assisted Reproductive Technology Programme

Materials and Methods

Trial Design

This trial had Clinical Trial Registration Number: NCT03228680 (clinicaltrials.gov).

This was a randomized, controlled, assessor-blind, multicenter, noninferiority trial of individualized follitropin delta dosing versus conventional follitropin beta dosing conducted at 17 investigational sites in Japan. The trial protocol (number 000273) was notified to the Pharmaceuticals and Medical Devices Agency (PMDA) and approved by the Institutional Review Boards covering all participating centers. The trial was performed in accordance with the principles of the Declaration of Helsinki, the International Council for Harmonisation Guidelines for Good Clinical Practice, Japanese Good Clinical Practice, and local regulatory requirements. All participants provided written, informed consent.

Population

Japanese women aged 20-40 years undergoing their first IVF/ICSI cycle and diagnosed with tubal infertility, unexplained infertility, infertility related to endometriosis stage I/II, or with partners diagnosed with male factor infertility were eligible for the trial. Additional main inclusion criteria were body mass index of 17.5-32.0 kg/m², regular menstrual cycles of 24-35 days, presence of both ovaries, and early follicular phase follicle-stimulating hormone (FSH) serum concentration of 1-15 IU/L. The main exclusion criteria were endometriosis stage Ill/IV, history of recurrent miscarriage, and use of hormonal preparations (except for thyroid medication) during the last menstrual cycle before randomization. There was no eligibility criterion limiting the serum antimullerian hormone (AMH) level at screening. All inclusion/exclusion criteria are listed in Supplemental Table 1 below.

Women were randomly assigned in a 1:1 ratio via a central computer-generated randomization sequence, prepared by an independent statistician. Randomization was stratified by center and according to AMH levels at screening (<15 pmol/L and >15 pmol/L) and performed in blocks of four within trial sites. All investigators, embryologists, and central laboratory personnel were blinded to treatment allocation throughout the trial.

Women randomized to follitropin delta (Rekovelle, 72 μg/2.16 mL, Ferring Pharmaceuticals) were given a fixed daily subcutaneous (SC) dose, determined by their serum AMH level at screening and body weight at randomization (AMH<15 pmol/L: 12 μg; AMH≥15 pmol/L: 0.10 to 0.19 μg/kg; the minimum daily dose was 6 μg; the maximum daily was 12 μg). The follitropin delta dosing algorithm (detailed in Supplemental Table 2) was programmed in the electronic case report form, which calculated the dose. The assigned daily dose was fixed throughout the stimulation period (i.e., no dose adjustments during stimulation).

Women randomized to follitropin beta (Follistim, 900 IU/1.08 mL, MSD K.K.) were administered a daily SC standard dose of 150 IU (expressed also as 15 μg of follitropin beta (19)) for the first 5 days, in line with the labelling (20) and international recommendations (21); thereafter, the dose could be adjusted up or down by 75 IU based on the individual response during stimulation as per the investigator's judgement, with 375 IU as the maximum daily dose allowed.

On day 2-3 of the menstrual cycle, women were randomized to COS with either follitropin delta or follitropin beta. To prevent a premature luteinizing hormone (LH) surge, a gonadotropin-releasing hormone (GnRH) antagonist (Ganirest, MSD K.K.) at a daily dose of 0.25 mg was initiated on day 6 and continued throughout the stimulation period. When ≥3 follicles with a diameter ≥17 mm were observed, triggering of final follicular maturation was performed with 5,000 IU urinary human chorionic gonadotropin (hCG FUJI, Fuji Pharma). In case of poor ovarian response (≥3 follicles with a diameter ≥17 mm could not be reached by day 20), the cycle was cancelled. In case of excessive ovarian response (≥25 follicles with a diameter 212 mm), women with 25-35 follicles with a diameter 212 mm could either be administered a GnRH agonist (600 μg Suprecur, 600 μg Buserecur, and 800 μg Nafarelil, as per local availability and at a dose according to site-specific procedures) or have the cycle cancelled as per the investigator's judgement, while the cycle was to be cancelled in case of >35 follicles with a diameter 212 mm.

Blood samples were collected during the trial for assessment of AMH, FSH, LH, estradiol, inhibin B, inhibin A, and progesterone. The serum concentration of AMH was measured at screening to assess eligibility and determine randomization strata. AMH was measured at a central laboratory using the automated Elecsys® AMH assay from Roche Diagnostics. Serum samples for assessment of endocrine parameters (FSH, LH, estradiol, inhibin B, inhibin A, and progesterone) were collected at start of stimulation, stimulation day 6, and end-of-stimulation, and analyzed at central laboratories.

Oocytes were retrieved 36h (±2h) after triggering of final follicular maturation and inseminated by IVF or ICSI, using ejaculated sperm from partner. The blastocyst of the best quality was transferred on day 5 after oocyte retrieval, while remaining blastocysts could be cryopreserved. For women who underwent triggering with GnRH agonist, no transfer was performed, and all blastocysts were cryopreserved.

Vaginal progesterone tablets (Lutinus, Ferring Pharmaceuticals) 100 mg three times daily were provided for luteal phase support from the day after oocyte retrieval until the day of the clinical pregnancy visit, if applicable. A phCG test was performed 13-15 days after blastocyst transfer (earlier assessment was accepted for women experiencing menses). Transvaginal ultrasound was performed 5-6 weeks after blastocyst transfer at the “clinical pregnancy visit” to assess clinical pregnancy (defined as at least one intrauterine or ectopic gestational sac) and vital pregnancy (defined as at least one intrauterine gestational sac with fetal heart beat). All pregnancies were followed until 4 weeks after live birth for information on pregnancy outcome including ongoing pregnancy (defined as at least one intrauterine viable fetus 10-11 weeks after blastocyst transfer) and neonatal health. Adverse events were recorded from the signed informed consent until the end-of-trial visit. Local tolerability of follitropin delta and follitropin beta following SC administration were assessed by the woman 3 times daily, i.e., immediately, 30 minutes, and 24 hours after each injection, and recorded in a diary. The injection site reactions (redness, itching, pain, swelling, and bruising) were assessed as none, mild, moderate, and severe.

Trial Outcomes

The primary endpoint was number of oocytes retrieved as a direct pharmacodynamic parameter of FSH action. The prespecified efficacy secondary endpoints included among others duration of stimulation, total gonadotropin dose, distribution of number of oocytes retrieved, extreme ovarian response in at-risk populations (defined as <4 oocytes retrieved for women with <15 pmol/L and >15 or 220 oocytes retrieved for women with ≥15 pmol/L), pregnancy outcomes, including clinical pregnancy as an important secondary endpoint, and live birth rates. Safety evaluations included adverse events, early and late OHSS, preventive interventions for early OHSS, cycle cancellation or blastocyst transfer cancellation due to excessive ovarian response/OHSS risk, and local tolerability. All cases of OHSS were categorized by grade (1, 2, 3, 4, or 5) and level (mild, moderate, or severe OHSS) according to Golan's classification system (22). Early OHSS was defined as onset ≤9 days after triggering of final follicular maturation and late OHSS as onset >9 days after triggering of final follicular maturation. Preventive interventions included cycle cancellation due to excessive ovarian response, triggering of final follicular maturation with GnRH agonist, or administration of dopamine agonist in women with ≥20 follicles of ≥12 mm.

Results

A total of 347 Japanese women were randomized and exposed, of which 170 were treated with individualized follitropin delta dosing and 177 with conventional follitropin beta dosing.

Dosing with individualized follitropin delta was noninferior to conventional follitropin beta with respect to number of oocytes retrieved. Overall, there was no difference between treatment groups in proportion of women with 8-14 oocytes retrieved (40.8% individualized follitropin delta versus 42.8% follitropin beta).

The incidence of OHSS was lower (all P<0.05) with individualized follitropin delta than with follitropin beta, including OHSS (early and late combined, 11.2% versus 19.8%), moderate/severe OHSS (7.1% versus 14.1%), OHSS and/or preventive interventions (11.8% versus 22.0%) and moderate/severe OHSS and/or preventive interventions (8.2% versus 17.5%). The incidence of early OHSS and/or preventive interventions for early OHSS was significantly (p<0.01) reduced from 20.9% with follitropin beta to 10.6% with follitropin delta.

Early Pregnancy Loss

Early pregnancy loss is generally defined as a pregnancy loss occurring before ongoing pregnancy. Herein, early pregnancy loss is defined as a pregnancy loss occurring between a positive pregnancy test performed 13-15 days after blastocyst transfer and a subsequent assessment of pregnancy (e.g. by transvaginal ultrasound) 10-11 weeks after blastocyst transfer. Early pregnancy losses between the phCG visit and a subsequent assessment of pregnancy (e.g. by transvaginal ultrasound) 10-11 weeks after blastocyst transfer in the trial are shown in the following Table:

| | |
|---|---|
| FE 999049 | FOLLISTIM |
| (N = 50) | (N = 52) |

Beta-hCG to vital pregnancy, n (%)

| | | | | |
|---|---|---|---|---|
| Loss | 10 | 20.0% | 14 | 26.9% |
| No loss | 40 | 80.0% | 38 | 73.1% |
| All | 50 | 100.0% | 52 | 100.0% |

Vital pregnancy to ongoing pregnancy, n (%)

| | | | | |
|---|---|---|---|---|
| Loss | 0 | | 4 | 10.5% |
| No loss | 40 | 100.0% | 34 | 89.5% |
| All | 40 | 100.0% | 38 | 100.0% |

Beta-hCG to ongoing pregnancy, n (%)

| | | | | |
|---|---|---|---|---|
| Loss | 10 | 20.0% | 18 | 34.6% |
| No loss | 40 | 80.0% | 34 | 65.4% |
| All | 50 | 100.0% | 52 | 100.0% |

Early pregnancy losses from the phCG visit to the clinical pregnancy visit were reported for 20.0% and 34.6% of subjects with a positive βhCG test in the Rekovelle and Follistim groups, respectively.

Live Birth Rates

Live birth is defined herein as “The birth of at least one live neonate”. The live birth rates for the two arms of the trial are shown in the following Table:

| FSH active | Live Birth Rate |
|---|---|
| Follitropin delta | 23.5% |
| (Rekovelle ®) | |
| Follitropin beta | 18.6% |
| (Follistim) | |

Thus, dosing with follitropin delta is associated with a markedly improved live birth rate compared to follitropin beta in this population. The improvement in live birth rate with Rekovelle (over Gonal F) for the Example 1 trial (see Table above) is 26.34%.

Conclusions

Thus, dosing with individualized follitropin delta in Japanese IVF/ICSI patients was superior to follitropin beta in terms of early pregnancy loss and live birth rate and indicated that fine-tuning of the gonadotropin dose resulting in modulation of the ovarian response in at-risk patients could positively influence clinical outcomes.

### Example 2

A similar trial in a Panasian (China, South Korea, Taiwan, Vietnam) patient population (1109 Patients) including Chinese patients comparing follitropin delta with follitropin alpha (CHO cell derived Gonal F) had similar results.

This was a randomised, controlled, assessor-blind trial conducted in Asian patients from mainland China, South Korea, Vietnam and Taiwan, undergoing their first IVF/ICSI cycle.

Randomisation was stratified by age (<35, 35-37, 38-40 years). The primary endpoint was ongoing pregnancy assessed 10-11 weeks after transfer (non-inferiority limit −10.0%; analysis adjusted for age strata). Patients <35 years underwent single embryo transfer if a good-quality embryo was available, otherwise double embryo transfer. Patients ≥35 years underwent double embryo transfer.

The primary objective of the trial was to demonstrate non-inferiority of FE 999049 compared with GONAL-F with respect to ongoing pregnancy rate in women undergoing controlled ovarian stimulation.

This was a randomised controlled trial using GONAL-F, an approved gonadotropin preparation, as active comparator. It was a parallel group design restricted to a single treatment cycle. The trial was open-label but assessor-blind. The trial was a multi-centre Pan-Asian trial. This set-up ensured that the required number of subjects could be recruited within a reasonable time and also had the advantage that it would facilitate subsequent generalisation of the results.

The trial was designed to demonstrate non-inferiority of FE 999049 versus GONAL-F with respect to ongoing pregnancy rate.

Subjects underwent controlled ovarian stimulation with an individualised dosing regimen of FE 999049 based on the subject's AMH level and body weight, or with a labelling recommended dosing regimen of GONAL-F, following a GnRH antagonist protocol. The daily FE 999049 (follitropin delta) dose was fixed throughout the stimulation period in this trial, and was identical to Example 1. The daily GONAL-F dose was fixed for the first five stimulation days, after which it could be adjusted by 75 IU per day based on the individual response, which was within the recommendations in the labelling. Monitoring of ovarian response by transvaginal ultrasound and blood sampling for assessment of several endocrine parameters was performed regularly during stimulation.

Oocytes were inseminated by either IVF or ICSI reflecting the procedures used in the target population for the proposed indication. Embryos were cultured for 3 days and embryo development was assessed from oocyte retrieval to the day of transfer, allowing evaluation of embryo development until cleavage stage. The duration of culture in this trial was adapted to clinical practice in Asia where transfer on day 3 after oocyte retrieval is most common.

The protocol required single or double embryo transfer on day 3 for all women, depending on age, with at least one good-quality embryo available.

Luteal phase support of the endometrium was provided via vaginal progesterone.

Subjects who achieved an ongoing pregnancy were followed to live birth to collect information on pregnancy outcome. In addition, neonatal health data are gathered at birth and at 4 weeks after birth.

The live birth rates for the two arms of the trial are shown in the following Table:

| | | | Live | Early |
|---|---|---|---|---|
| | | | Birth | Pregnancy |
| | Live | Early | Rate | Loss |
| | Birth | Pregnancy | (Chinese | (Chinese |
| FSH active | Rate | Loss | Patients) | Patients) |
| Follitropin delta | 31.3% * | 25.0% | 31.0% | 25.9% |
| (Rekovelle) | | | | |
| Follitropin alpha | 24.7% | 27.2% | 25.5% | 29.0% |
| (Gonal F) | | | | |

* P < 0.05

Thus, dosing with follitropin delta is associated with a markedly improved live birth rate and reduced early pregnancy loss compared to follitropin alpha in this population. The improvement in live birth rate with Rekovelle (over Gonal F) for whole population in the Example 2 trial (see Table above) is 26.72%.

The ongoing pregnancy rate was 31.3% with follitropin delta (Rekovelle) and 25.7% with follitropin alfa (Gonal F) (adjusted difference 5.4% [95% Cl: −0.2%; 11.0%]). The live birth rate was significantly higher at 31.3% with follitropin delta compared to 24.7% with follitropin alfa (adjusted difference 6.4% [95% Cl: 0.9%; 11.9%]; p<0.05). Live birth rates per age stratum were as follows for follitropin delta and follitropin alfa, respectively; <35 years: 31.0% versus 25.0%, 35-37 years: 35.3% versus 26.7%, 38-40 years: 20.0% versus 14.3%. The 38-40 year age group have a 39.8% improvement in live birth with follitropin delta (20% vs 14.3%), while the 35-37 year age group have a 32.2% improvement (35.3% vs 26.7%) and the <35 year age group have a 24% improvement (31.0% vs 25.0%). Thus, the improvement in live birth rate seen with follitropin delta is greater in the higher age strata.

For the Example 2 PanAsia trial, the live birth rate for patients treated with Rekovelle (follitropin delta) and Gonal F (follitropin alpha) stratified by age. The relative difference between Rekovelle and Gonal-F (that is, the improvement in live birth rate with Rekovelle compared to Gonal F) for the whole population in the Example 1 and Example 2 trials are 26.34% and 26.72% respectively. The applicants found that, in the PanAsia trial of Example 2, the relative difference between Rekovelle and Gonal-F begins to markedly increase above the value for the whole population (26.72%) in patients of age 28 or greater (relative difference 31.4% compared to 26.72%) and is particularly marked in patients age 30 or greater (51.2% compared to 26.72%), supporting the contention that improvement in live birth rate seen with follitropin delta is greater in patients age 28 and above, and is even more improved in patients age 30 and above.

The incidence of early OHSS and/or preventive interventions for early OHSS was significantly (p<0.01) reduced from 9.6% with follitropin alfa to 5.0% with follitropin delta.

The number of oocytes retrieved was 10.0±6.1 with follitropin delta and 12.4±7.3 with follitropin alfa. Individualised follitropin delta dosing compared to conventional follitropin alfa dosing resulted in 2 more oocytes (9.6±5.3 versus 7.6±3.5) in potential low responders (AMH<15 pmol/L) and 3 fewer oocytes (10.1 6.3 versus 13.8±7.5) in potential high responders (AMH≥15 pmol/L). Among patients with AMH≥15 pmol/L, excessive response occurred less frequently with individualised than conventional dosing (≥15 oocytes: 20.2% versus 39.1%; ≥20 oocytes: 6.7% versus 18.5%).

Total gonadotropin dose was reduced from 109.9±32.9 μg with follitropin alfa to 77.5±24.4 μg with follitropin delta.

Summary

In a trial comparing REKOVELLE® with a CHO cell derived rFSH, the applicants unexpectedly found that early pregnancy loss was reported for 20% of subjects with a positive βhCG test treated with REKOVELLE®, compared to 34.6% of subjects with a positive βhCG test treated with the CHO cell derived rFSH (Example 1). Similar results were found in a similar Pan Asian trial, in an Asian (Chinese, Taiwanese, Vietnamese and South Korean) patient population (Example 2), and it is believed that the effect, shown in these large scale Phase Ill clinical trials, will be shown for all patient populations. Similarly, these Japanese and Pan Asian trials have shown that REKOVELLE® treatment increases the probability of a live birth, compared to treatment with a CHO cell derived rFSH, with this effect being more marked in older patients.

Example 1 Supplementary Table 1 Eligibility criteria
Inclusion criteria

| | |
|---|---|
| 1. | Informed consent documents signed prior to any trial-related procedures. |
| 2. | In good physical and mental health. |
| 3. | Japanese females between the ages of 20 and 40 years. The subjects must be at least |
| | 20 years (including the 20^{th} birthday) when they sign the informed consent documents |
| | and no more than 40 years (up to the day before the 41^{st} birthday) at the time of |
| | randomization. |
| 4. | Infertile women diagnosed with tubal infertility, unexplained infertility, endometriosis |
| | stage I/II (defined by the revised American Society for Reproductive Medicine (ASRM) |
| | classification), or with partners diagnosed with male factor infertility, eligible for in vitro |
| | fertilization (IVF) and/or intracytoplasmic sperm injection (ICSI) treatment using |
| | ejaculated sperm from male partner. |
| 5. | Infertility for at least 1 year before randomization (not applicable in case of tubal or |
| | severe male factor infertility). |
| 6. | The trial cycle will be the subject's first controlled ovarian stimulation cycle for IVF/ICSI. |
| 7. | Regular menstrual cycles of 24-35 days (both inclusive), presumed to be ovulatory. |
| 8. | Hysterosalpingography, hysteroscopy, saline infusion sonography or transvaginal |
| | ultrasound documenting a uterus consistent with expected normal function (e.g., no |
| | evidence of clinically interfering uterine fibroids defined as submucous or intramural |
| | fibroids larger than 3 cm in diameter, no polyps, and no congenital structural |
| | abnormalities which are associated with a reduced chance of pregnancy) within 1 year |
| | prior to screening. This also includes women who have been diagnosed with any of the |
| | above medical conditions but have had them surgically corrected within 1 year prior to |
| | screening. |
| 9. | Transvaginal ultrasound documenting presence and adequate visualization of both |
| | ovaries, without evidence of significant abnormality (e.g., no endometrioma greater than |
| | 3 cm or enlarged ovaries which would contraindicate the use of gonadotropins) and |
| | fallopian tubes and surrounding tissue without evidence of significant abnormality (e.g., |
| | no hydrosalpinx) within 1 year prior to screening. Both ovaries must be accessible for |
| | oocyte retrieval. |
| 10. | Early follicular phase (cycle day 2-4) serum levels of FSH between 1 and 15 IU/L (results |
| | obtained within 3 months prior to screening). |
| 11. | Negative serum hepatitis B surface antigen (HBsAg), hepatitis C virus (HCV), and human |
| | immunodeficiency virus (HIV) antibody tests within 1 year prior to screening. |
| 12. | Body mass index (BMI) between 17.5 and 32.0 kg/m² (both inclusive) at screening. |
| 13. | Willing to accept transfer of one blastocyst. |

Exclusion criteria

| | |
|---|---|
| 1. | Known endometriosis stage III/IV (defined by the revised ASRM classification). |
| 2. | One or more follicles >10 mm (including cysts) observed on the transvaginal ultrasound |
| | prior to start of stimulation on stimulation day 1 (puncture of cysts prior randomization is |
| | allowed). |
| 3. | Known history of recurrent miscarriage (defined as three consecutive losses after |
| | ultrasound confirmation of pregnancy (excl. ectopic pregnancy) and before week 24 of |
| | pregnancy). |
| 4. | Known abnormal karyotype of subject or of her partner. In case the sperm production is |
| | severely impaired (concentration <1 million/mL), normal karyotype, including no Y- |
| | chromosome microdeletion, must be documented. |
| 5. | Active arterial or venous thromboembolism or severe thrombophlebitis, or a history of |
| | these events. |
| 6. | Known porphyria. |
| 7. | Any known clinically significant systemic disease (e.g., insulin-dependent diabetes). |
| 8. | Known inherited or acquired thrombophilia disease. |
| 9. | Any known endocrine or metabolic abnormalities (pituitary, adrenal, pancreas, liver, or |
| | kidney) which can compromise participation in the trial with the exception of controlled |
| | thyroid function disease. |
| 10. | Known presence of anti-FSH antibodies (based on the information available in the |
| | subject's medical records). |
| 11. | Known tumors of the ovary, breast, uterus, adrenal gland, pituitary, or hypothalamus |
| | which would contraindicate the use of gonadotropins. |
| 12. | Any abnormal finding of clinical chemistry, hematology, or vital signs at screening, which |
| | is judged clinically relevant by the investigator. |
| 13. | Known moderate or severe impairment of renal or hepatic function. |
| 14. | Currently breast-feeding. |
| 15. | Undiagnosed vaginal bleeding. |
| 16. | Known abnormal cervical cytology of clinical significance observed within 3 years prior to |
| | screening (unless the clinical significance has been resolved). |
| 17. | Findings from the laboratory analyses at screening which preclude gonadotropin |
| | stimulation. |
| 18. | Findings at the gynecological examination at screening which preclude gonadotropin |
| | stimulation. |
| 19. | Findings at the gynecological examination at screening which are associated with a |
| | reduced chance of pregnancy, e.g., congenital uterine abnormalities or retained |
| | intrauterine device. |
| 20. | Pregnancy (must be confirmed by negative urinary pregnancy tests at screening and |
| | prior to randomization) or contraindication to pregnancy. |
| 21. | Known current active pelvic inflammatory disease. |
| 22. | Use of hormonal preparations (except for thyroid medication) or fertility modifiers during |
| | the last menstrual cycle before randomization, including dehydroepiandrosterone |
| | (DHEA), metformin, and cycle programming with oral contraceptives, progestogen, or |
| | estrogen preparations. |
| 23. | Known history of chemotherapy (except for gestational conditions) or radiotherapy. |
| 24. | Current or past (1 year prior to randomization) abuse of alcohol or drugs, and/or current |
| | (last month) intake of more than 14 units of alcohol per week. |
| 25. | Current or past (3 months prior to randomization) smoking habit of more than 10 |
| | cigarettes per day. |
| 26. | Hypersensitivity to any drug substance or excipients in the medicinal products used in |
| | the trial. |
| 27. | Hypersensitivity to any drug substance or excipients in a GnRH or any GnRH analogue/ |
| | derivative. |
| 28. | Previous participation in the trial. |
| 29. | Current participation in another trial, including follow-up period. |
| 30. | Use of any non-registered investigational drugs during the last 3 months prior to |
| | screening. |

FSH: follicle-stimulating hormone; GnRH: gonadotropin-releasing hormone; IU: international unit(s).

Example 1 Supplementary Table 2 Individualized follitropin delta dosing regimen

| AMH (pmol/L) | <15 | 15-16 | 17 | 18 | 19-20 | 21-22 | 23-24 | 25-27 | 28-32 | 33-39 | >40 |
|---|---|---|---|---|---|---|---|---|---|---|---|
| Fixed daily dose | 12 μg | 0.19 | 0.18 | 0.17 | 0.16 | 0.15 | 0.14 | 0.13 | 0.12 | 0.11 | 0.10 |
| of follitropin delta | | | | | | | | | | | |
| (μg/kg) | | | | | | | | | | | |

AMH concentration was rounded off to the nearest integers before determination of dose.
With the exception of 12 μg for AMH <15 pmol/L, all doses are expressed as μg/kg.
Minimum daily dose was 6 μg.
Maximum daily dose was 12 μg.
AMH: antimüllerian hormone.

There have been disclosed hereinbefore the compositions, compositions for use, uses and methods defined by the following numbered paragraphs:

1. A composition comprising recombinant follicle stimulating hormone (FSH) for use in reducing the likelihood of early pregnancy loss in a patient treated for infertility by controlled ovarian stimulation, wherein the recombinant FSH includes α2,3- and α2,6-sialylation (e.g. wherein 1% to 60% of the total sialylation is α2,6-sialylation and wherein 40% to 99% of the total sialylation is α2,3-sialylation, e.g. wherein 5% to 20% of the total sialylation is α2,6-sialylation and wherein 80% to 95% of the total sialylation is α2,3-sialylation).

2. A composition comprising recombinant follicle stimulating hormone (FSH) for use in increasing the probability of live birth following treatment for infertility by controlled ovarian stimulation, wherein the recombinant FSH includes α2,3- and α2,6-sialylation (e.g. wherein 1% to 60% of the total sialylation is α2,6-sialylation and wherein 40% to 99% of the total sialylation is α2,3-sialylation, e.g. wherein 5% to 20% of the total sialylation is α2,6-sialylation and wherein 80% to 95% of the total sialylation is α2,3-sialylation).

3. A composition for use according to paragraph 1 or 2 wherein the patient has serum AMH level of <15 pmol/L, wherein the composition is to be administered at a dose of, or equivalent to, 11 to 13 μg recombinant FSH per day.

4. A composition for use according to paragraph 3 wherein the use comprises a step of determining the serum AMH level of the patient, and a step of administering the dose to a patient having serum AMH level of <15 pmol/L.

5. A composition for use according to paragraph 1 or 2 wherein the patient has serum AMH level of ≥15 pmol/L, wherein the composition is to be administered at a dose of, or equivalent to, 0.09 to 0.19 μg recombinant FSH per kg bodyweight of the patient per day.

6. A composition for use according to paragraph 5 wherein the use comprises a step of determining the serum AMH level of the patient, and a step of administering the dose to a patient having serum AMH level of ≥15 pmol/L.

7. A composition for use according to any preceding paragraph wherein the patient is (for example is identified as being) a female of Japanese ethnicity, for example a female with two ethnic Japanese parents.

8. A composition for use according to any preceding paragraph wherein the patient is (for example is identified as being) a female of Asian (e.g. Chinese, Taiwanese, Vietnamese or South Korean) ethnicity, for example a patient with two ethnic Asian (e.g. Chinese, Taiwanese, Vietnamese or South Korean) parents.

9. A composition for use according to any preceding paragraph wherein the recombinant FSH includes α2,3- and α2,6-sialylation wherein 5% to 20% of the total sialylation is α2,6-sialylation and wherein 80% to 95% of the total sialylation is α2,3-sialylation.

10. A composition for use according to any preceding paragraph wherein the recombinant FSH is recombinant FSH which has been produced or expressed in a human cell line.

11. A method of reducing the likelihood of early pregnancy loss in a patient treated for infertility by controlled ovarian stimulation, the method comprising administering to the patient a pharmaceutically effective amount of a composition comprising recombinant FSH; wherein the recombinant FSH includes α2,3- and α2,6-sialylation (e.g. wherein 1% to 60% of the total sialylation is α2,6-sialylation and wherein 40% to 99% of the total sialylation is α2,3-sialylation, e.g. wherein 5% to 20% of the total sialylation is α2,6-sialylation and wherein 80% to 95% of the total sialylation is α2,3-sialylation).

12. A method of increasing the probability of live birth following treatment of a patient for infertility by controlled ovarian stimulation, the method comprising administering to the patient a pharmaceutically effective amount of a composition comprising recombinant FSH; wherein the recombinant FSH includes α2,3- and α2,6-sialylation (e.g. wherein 5% to 20% of the total sialylation is α2,6-sialylation and wherein 80% to 95% of the total sialylation is α2,3-sialylation).

13. A method according to paragraph 11 or 12 wherein the patient has serum AMH level of <15 pmol/L, wherein the composition is to be administered at a dose of, or equivalent to, 11 to 13 μg recombinant FSH per day.

14. A method according to paragraph 13 comprising a step of determining the serum AMH level of the patient, and a step of administering the dose to a patient having serum AMH level of <15 pmol/L.

15. A method according to paragraph 11 or 12 wherein the patient has serum AMH level of ≥15 pmol/L, wherein the composition is to be administered at a dose of, or equivalent to, 0.09 to 0.19 μg recombinant FSH per kg bodyweight of the patient per day.

16. A method according to paragraph 15 wherein the method comprises a step of determining the serum AMH level of the patient, and a step of administering the dose to a patient having serum AMH level of ≥15 pmol/L.

17. A method according to any of paragraphs 11 to 16 wherein the patient is (for example is identified as being) a female of Japanese ethnicity, for example a female with two ethnic Japanese parents.

18. A method according to any of paragraphs 11 to 16 wherein the patient is (for example is identified as being) a female of Asian (e.g. Chinese, Taiwanese, Vietnamese or South Korean) ethnicity, for example a patient with two ethnic Asian (e.g. Chinese, Taiwanese, Vietnamese or South Korean) parents.

19. A method according to any of paragraphs 11 to 18 wherein the recombinant FSH includes α2,3- and α2,6-sialylation wherein 5% to 20% of the total sialylation is α2,6-sialylation and wherein 80% to 95% of the total sialylation is α2,3-sialylation.

20. A method according to any of paragraphs 11 to 19 wherein the recombinant FSH is recombinant FSH which has been produced or expressed in a human cell line.

21. A composition comprising recombinant follicle stimulating hormone (FSH) for use in the treatment of infertility in a female patient of age 35 years or greater, for example of age 36 years or greater, for example of age 37 years or greater, for example of age 38 years or greater, for example a female patient of age 38-40 years, to increase the probability of live birth, wherein the recombinant FSH includes α2,3- and α2,6-sialylation (e.g. wherein 1% to 60% of the total sialylation is α2,6-sialylation and wherein 40% to 99% of the total sialylation is α2,3-sialylation, e.g. wherein 5% to 20% of the total sialylation is α2,6-sialylation and wherein 80% to 95% of the total sialylation is α2,3-sialylation).

22. A composition for use according to paragraph 21 wherein the use comprises a step of determining the age of the patient, and a step of administering the recombinant FSH to a patient of age 35 years or greater, for example of age 38 years or greater, for example a female patient of age 38-40 years.

23. A composition for use according to paragraph 21 or 22 wherein the patient has serum AMH level of <15 pmol/L, wherein the composition is to be administered at a dose of, or equivalent to, 11 to 13 μg recombinant FSH per day.

24. A composition for use according to paragraph 23 wherein the use comprises a step of determining the serum AMH level of the patient, and a step of administering the dose to a patient having serum AMH level of <15 pmol/L.

25. A composition for use according to paragraph 21 or 22 wherein the patient has serum AMH level of ≥15 pmol/L, wherein the composition is to be administered at a dose of, or equivalent to, 0.09 to 0.19 μg recombinant FSH per kg bodyweight of the patient per day.

26. A composition for use according to paragraph 25 wherein the use comprises a step of determining the serum AMH level of the patient, and a step of administering the dose to a patient having serum AMH level of ≥15 pmol/L.

27. A composition for use according to any preceding paragraph wherein the patient is (for example is identified as being) a female of Japanese ethnicity, for example a female with two ethnic Japanese parents.

28. A composition for use according to any preceding paragraph wherein the patient is (for example is identified as being) a female of Asian (e.g. Chinese, Taiwanese, Vietnamese or South Korean) ethnicity, for example a patient with two ethnic Asian (e.g. Chinese, Taiwanese, Vietnamese or South Korean) parents.

29. A composition for use according to any preceding paragraph wherein the recombinant FSH includes α2,3- and α2,6-sialylation wherein 5% to 20% of the total sialylation is α2,6-sialylation and wherein 80% to 95% of the total sialylation is α2,3-sialylation.

30. A composition for use according to any preceding paragraph wherein the recombinant FSH is recombinant FSH which has been produced or expressed in a human cell line.

31. A method of increasing the probability of live birth following treatment of infertility (e.g. by controlled ovarian stimulation) of a female patient of age 35 years or greater, for example of age 36 years or greater, for example of age 37 years or greater, for example of age 38 years or greater, for example a female patient of age 38-40 years, the method comprising administering to the patient a pharmaceutically effective amount of a composition comprising recombinant FSH; wherein the recombinant FSH includes α2,3- and α2,6-sialylation (e.g. wherein 5% to 20% of the total sialylation is α2,6-sialylation and wherein 80% to 95% of the total sialylation is α2,3-sialylation).

32. A method according to paragraph 31 comprising a step of determining the age of the patient, and a step of administering the pharmaceutically effective amount of the composition comprising recombinant FSH to a patient of age 35 years or greater, for example of age 36 years or greater, for example of age 37 years or greater, for example of age 38 years or greater, for example a female patient of age 38-40 years.

33. A method of treatment of infertility (e.g. by controlled ovarian stimulation, e.g. to increase the probability of live birth) in a female patient of age 35 years or greater, for example of age 36 years or greater, for example of age 37 years or greater, for example of age 38 years or greater, for example a female patient of age 38-40 years, the method comprising a step of determining the age of the patient, and a step of administering a pharmaceutically effective amount of a composition comprising recombinant FSH to a patient of age 35 years or greater, for example of age 36 years or greater, for example of age 37 years or greater, for example of age 38 years or greater, for example a female patient of age 38-40 years; wherein the recombinant FSH includes α2,3- and α2,6-sialylation (e.g. wherein 5% to 20% of the total sialylation is α2,6-sialylation and wherein 80% to 95% of the total sialylation is α2,3-sialylation).

34. A method according to paragraph 31, 32 or 33 wherein the patient has serum AMH level of <15 pmol/L, wherein the composition is to be administered at a dose of, or equivalent to, 11 to 13 μg recombinant FSH per day, the method optionally comprising a step of determining the serum AMH level of the patient, and a step of administering the dose to a patient having serum AMH level of <15 pmol/L; or a method according to any of paragraphs 31 to 33 wherein the patient has serum AMH level of ≥15 pmol/L, wherein the composition is to be administered at a dose of, or equivalent to, 0.09 to 0.19 μg recombinant FSH per kg bodyweight of the patient per day, the method optionally comprising a step of determining the serum AMH level of the patient, and a step of administering the dose to a patient having serum AMH level of ≥15 pmol/L.

35. A method according to any of paragraphs 31 to 34 wherein the patient is (for example is identified as being) a female of Japanese ethnicity, for example a female with two ethnic Japanese parents; or wherein the patient is (for example is identified as being) a female of Asian (e.g. Chinese, Taiwanese, Vietnamese or South Korean) ethnicity, for example a patient with two ethnic Asian (e.g. Chinese, Taiwanese, Vietnamese or South Korean) parents; and/or wherein the recombinant FSH includes α2,3- and α2,6-sialylation wherein 5% to 20% of the total sialylation is α2,6-sialylation and wherein 80% to 95% of the total sialylation is α2,3-sialylation; and/or wherein the recombinant FSH is recombinant FSH which has been produced or expressed in a human cell line.

36. A composition comprising recombinant follicle stimulating hormone (FSH) for use in the treatment of infertility in a female patient of age 28 years or greater, for example of age 30 years or greater, for example of age 31 years or greater, for example of age 32 years or greater, for example of age 33 years or greater, for example of age 35 years or greater, for example of age 36 years or greater, for example of age 37 years or greater, for example of age 38 years or greater, for example a female patient of age 28-40 years, for example a female patient of age 30-40 years, to increase the probability of live birth, wherein the recombinant FSH includes α2,3- and α2,6-sialylation (e.g. wherein 1% to 60% of the total sialylation is α2,6-sialylation and wherein 40% to 99% of the total sialylation is α2,3-sialylation, e.g. wherein 5% to 20% of the total sialylation is α2,6-sialylation and wherein 80% to 95% of the total sialylation is α2,3-sialylation).

37. A composition for use according to paragraph 36 wherein the use comprises a step of determining the age of the patient, and a step of administering the recombinant FSH to a patient of age 28 years or greater, for example of age 30 years or greater, for example a female patient of age 28-40 years, for example a female patient of age 30-40 years.

38. A composition for use according to paragraph 36 or 37 wherein the patient has serum AMH level of <15 pmol/L, wherein the composition is to be administered at a dose of, or equivalent to, 11 to 13 μg recombinant FSH per day.

39. A composition for use according to paragraph 38 wherein the use comprises a step of determining the serum AMH level of the patient, and a step of administering the dose to a patient having serum AMH level of <15 pmol/L.

40. A composition for use according to paragraph 36 or 37 wherein the patient has serum AMH level of ≥15 pmol/L, wherein the composition is to be administered at a dose of, or equivalent to, 0.09 to 0.19 μg recombinant FSH per kg bodyweight of the patient per day.

41. A composition for use according to paragraph 40 wherein the use comprises a step of determining the serum AMH level of the patient, and a step of administering the dose to a patient having serum AMH level of ≥15 pmol/L.

42. A composition for use according to any of paragraphs 36 to 41 wherein the patient is (for example is identified as being) a female of Japanese ethnicity, for example a female with two ethnic Japanese parents.

43. A composition for use according to any of paragraphs 36 to 42 wherein the patient is (for example is identified as being) a female of Asian (e.g. Chinese, Taiwanese, Vietnamese or South Korean) ethnicity, for example a patient with two ethnic Asian (e.g. Chinese, Taiwanese, Vietnamese or South Korean) parents.

44. A composition for use according to any of paragraphs 36 to 43 wherein the recombinant FSH includes α2,3- and α2,6-sialylation wherein 5% to 20% of the total sialylation is α2,6-sialylation and wherein 80% to 95% of the total sialylation is α2,3-sialylation.

45. A composition for use according to any of paragraphs 36 to 44 wherein the recombinant FSH is recombinant FSH which has been produced or expressed in a human cell line.

46. A method of increasing the probability of live birth following treatment of infertility (e.g. by controlled ovarian stimulation) of a female patient of age 28 years or greater, for example of age 30 years or greater, for example of age 31 years or greater, for example of age 32 years or greater, for example of age 33 years or greater, for example of age 35 years or greater, for example of age 36 years or greater, for example of age 37 years or greater, for example of age 38 years or greater, for example a female patient of age 28-40 years, for example a female patient of age 30-40 years, the method comprising administering to the patient a pharmaceutically effective amount of a composition comprising recombinant FSH; wherein the recombinant FSH includes α2,3- and α2,6-sialylation (e.g. wherein 5% to 20% of the total sialylation is α2,6-sialylation and wherein 80% to 95% of the total sialylation is α2,3-sialylation).

47. A method according to paragraph 46 comprising a step of determining the age of the patient, and a step of administering the pharmaceutically effective amount of the composition comprising recombinant FSH to the patient of age 28 years or greater, for example of age 30 years or greater, for example of age 31 years or greater, for example of age 32 years or greater, for example of age 33 years or greater, for example of age 35 years or greater, for example of age 36 years or greater, for example of age 37 years or greater, for example of age 38 years or greater, for example a female patient of age 28-40 years, for example a female patient of age 30-40 years.

48. A method of treatment of infertility (e.g. by controlled ovarian stimulation, e.g. to increase the probability of live birth) in a female patient of age 28 years or greater, for example of age 30 years or greater, for example of age 31 years or greater, for example of age 32 years or greater, for example of age 33 years or greater, for example of age 35 years or greater, for example of age 36 years or greater, for example of age 37 years or greater, for example of age 38 years or greater, for example a female patient of age 28-40 years, for example a female patient of age 30-40 years, the method comprising a step of determining the age of the patient, and a step of administering a pharmaceutically effective amount of a composition comprising recombinant FSH to the patient of age 28 years or greater, for example of age 30 years or greater, for example of age 31 years or greater, for example of age 32 years or greater, for example of age 33 years or greater, for example of age 35 years or greater, for example of age 36 years or greater, for example of age 37 years or greater, for example of age 38 years or greater, for example a female patient of age 28-40 years, for example a female patient of age 30-40 years; wherein the recombinant FSH includes α2,3- and α2,6-sialylation (e.g. wherein 5% to 20% of the total sialylation is α2,6-sialylation and wherein 80% to 95% of the total sialylation is α2,3-sialylation).

49. A method according to paragraph 46, 47 or 48 wherein the patient has serum AMH level of <15 pmol/L, wherein the composition is to be administered at a dose of, or equivalent to, 11 to 13 μg recombinant FSH per day, the method optionally comprising a step of determining the serum AMH level of the patient, and a step of administering the dose to a patient having serum AMH level of <15 pmol/L; or a method according to any of paragraphs 11 to 13 wherein the patient has serum AMH level of ≥15 pmol/L, wherein the composition is to be administered at a dose of, or equivalent to, 0.09 to 0.19 μg recombinant FSH per kg bodyweight of the patient per day, the method optionally comprising a step of determining the serum AMH level of the patient, and a step of administering the dose to a patient having serum AMH level of ≥15 pmol/L.

50. A method according to any of paragraphs 46 to 49 wherein the patient is (for example is identified as being) a female of Japanese ethnicity, for example a female with two ethnic Japanese parents; or wherein the patient is (for example is identified as being) a female of Asian (e.g. Chinese, Taiwanese, Vietnamese or South Korean) ethnicity, for example a patient with two ethnic Asian (e.g. Chinese, Taiwanese, Vietnamese or South Korean) parents; and/or wherein the recombinant FSH includes α2,3- and α2,6-sialylation wherein 5% to 20% of the total sialylation is α2,6-sialylation and wherein 80% to 95% of the total sialylation is α2,3-sialylation; and/or wherein the recombinant FSH is recombinant FSH which has been produced or expressed in a human cell line.

1-20. (canceled)

21. A method for increasing the probability of live birth in the treatment of infertility in a female patient of age 30 to 40 years, comprising administering a composition comprising recombinant follicle stimulating hormone (FSH) to the patient, wherein the recombinant FSH includes α2,3- and α2,6-sialylation.

22. The method of claim 21, further comprising determining the age of the patient prior to the administering, wherein the patient is of age 30 to 40 years.

23. The method of claim 21, wherein the patient is of age 30 to 37 years.

24. The method of claim 23, further comprising determining the age of the patient prior to the administering.

25. The method of claim 21, wherein the patient is of age 36 to 40 years.

26. The method of claim 25, further comprising determining the age of the patient prior to the administering.

27. The method of claim 21, wherein the patient has a serum AMH level of <15 pmol/L, and the composition is administered at a dose of, or equivalent to, 11 to 13 μg recombinant FSH per day.

28. The method of claim 27, further comprising determining the serum AMH level of the patient prior to the administering.

29. The method of claim 21, wherein the patient has a serum AMH level of ≥15 pmol/L, and the composition is administered at a dose of, or equivalent to, 0.09 to 0.19 μg recombinant FSH per kg bodyweight of the patient per day.

30. The method of claim 29, further comprising determining the serum AMH level of the patient prior to the administering.

31. The method of claim 21, wherein the patient is of age 35 to 40 years, wherein if the patient has a serum AMH level of <15 pmol/L, the composition is administered at a dose of, or equivalent to, 11 to 13 μg recombinant FSH per day, and if the patient has serum AMH level of ≥15 pmol/L, the composition is administered at a dose of, or equivalent to, 0.09 to 0.19 μg recombinant FSH per kg bodyweight of the patient per day.

32. The method of claim 21, wherein the patient is of age 35 to 40 years, further comprising:
determining the age of the patient prior to the administering; and
determining the serum AMH level of the patient prior to the administering, and if the patient has a serum AMH level of <15 pmol/L, administering the composition at a dose of, or equivalent to, 11 to 13 μg recombinant FSH per day, and if the patient has serum AMH level of ≥15 pmol/L, administering the composition at a dose of, or equivalent to, 0.09 to 0.19 μg recombinant FSH per kg bodyweight of the patient per day.

33. The method of claim 21, wherein the patient is of an ethnicity selected from Japanese, Chinese, Taiwanese, Vietnamese, and South Korean.

34. The method of claim 33, wherein the patient is of age 35 to 40 years, and the method further comprises determining the age of the patient prior to the administering.

35. A method for reducing the likelihood of early pregnancy loss in a female patient treated for infertility by controlled ovarian stimulation, comprising administering to the patient a composition comprising recombinant follicle stimulating hormone (FSH), wherein the recombinant FSH includes α2,3- and α2,6-sialylation.

36. A method for increasing the probability of live birth following treatment of a female patient for infertility by controlled ovarian stimulation, comprising administering to the patient a composition comprising recombinant FSH that includes α2,3- and α2,6-sialylation, wherein the patient has a serum AMH level of ≥15 pmol/L, and wherein the composition is administered at a dose of, or equivalent to, 0.09 to 0.19 μg recombinant FSH per kg bodyweight of the patient per day.

37. The method according to claim 21, wherein 1% to 60% of the total sialylation of the recombinant FSH is α2,6-sialylation and wherein 40% to 99% of the total sialylation of the recombinant FSH is α2,3-sialylation.

38. The method according to claim 21, wherein 5% to 20% of the total sialylation of the recombinant FSH is α2,6-sialylation and wherein 80% to 95% of the total sialylation of the recombinant FSH is α2,3-sialylation.

39. The method according to claim 21, wherein the recombinant FSH is recombinant FSH which has been produced or expressed in a human cell line.
